(19) United States
(12) Patent Application Publication
(10) Pub. No.: US 2010/0028450 A1
(43) Pub. Date: Feb. 4, 2010

(54) TOLEROGENIC BIODEGRADABLE ARTIFICIAL ANTIGEN PRESENTING SYSTEM

(75) Inventor: Chenthamarakshan Vasu, Bensenville, IL (US)

Correspondence Address: BARNES & THORNBURG LLP, P.O. BOX 2786, CHICAGO, IL, 60690-2786, US

(73) Assignee: THE BOARD OF TRUSTEES OF THE UNIVERSITY OF ILLINOI S, Urbana, IL (US)

(21) Appl. No.: 12/162,292

(22) PCT Filed: Jan. 25, 2007
(86) PCT No.: PCT/US07/01867

§ 371 Date: Jul. 25, 2008

Related U.S. Application Data
(60) Provisional application No. 60/762,092, filed on Jan. 25, 2006.

(51) Int. Cl. A61K 9/14; A61K 39/00; A61K 35/12; C12N 5/00; A61P 37/00

(52) U.S. Cl. 424/491; 424/184.1; 424/93.71; 435/375; 435/377

(57) ABSTRACT

An artificial antigen presenting system is presented. The herein presented microspheres combine negative regulators individually or at varying combinations along with MCH molecules and can induce antigen specific tolerance. The herein described methods provide for the construction of artificial biodegradable microsomes containing MHC: peptide complexes, accessory molecules, co-stimulatory molecules, adhesion molecules, and other molecules relevant to T cell binding or modulation. Additionally, the present invention is directed to compositions and methods for treating conditions which would benefit from modulation of T cell response, for example, autoimmune disorders, allergies, cancers, viral infections, and graft rejection.

### CROSS REFERENCE TO RELATED APPLICATIONS

This application claims the benefit of U.S. Provisional Application 60/762,092 filed on Jan. 25, 2006.

### GOVERNMENT RIGHTS

This work was supported by NIH Grant No. R21A1069848-01. The U.S. Government has certain rights in this invention.

### BACKGROUND OF THE INVENTION

T cell activation was once thought to be involved mainly with TCR ligation by cognate MHC molecules. However, recent identification and characterization of several signaling pathways related to T cell activation and homeostasis have significantly changed T cell immunology research. Co-stimulatory and co-inhibitory pathways in the B7:CD28 super family play key roles in regulating T cell activation and tolerance and are being exploited as therapeutic targets for treating various immune mediated disorders/conditions. Greenwald R J, Freeman G J, Sharpe A H. The B7 family revisited. Annu Rev Immunol. 2005; 23:515-48. These pathways are vital not only in providing positive second signals that promote and sustain T cell responses, but also in inhibitory signals that downregulate T cell responses. Chen LP. 2004. Co-inhibitory molecules of the B7-CD28 family in the control of T-cell immunity. Nat. Rev. Immunol. 4:336 47. These inhibitory signals control, terminate, or attenuate T cell responses, and appear to be very critical for regulating T cell tolerance and autoimmunity. The B7-1/B7-2:CD28/CTLA-4 pathway is the most thoroughly characterized T cell co-stimulatory/inhibitory pathway. It is highly complex because of the dual specificity of ligands B7-1 (CD80) and B7-2 (CD86) for the stimulator CD28 and the inhibitor CTLA-4 (CD152). Sharpe A H, Freeman G J. 2002. The B7-CD28 superfamily. Nat. Rev. Immunol. 2:116 26; Greenwald R J, Latchman Y E, Sharpe A H. 2002. Negative co-receptors on lymphocytes. Curr. Opin. Immunol. 14:391 96; Chikuma S, Bluestone J A. 2003. CTLA-4 and tolerance: the biochemical point of view. Immunol. Res. 28:241 53.

Recently, two new pathways in the B7:CD28 family: one involving a stimulatory ICOS and ICOS-L, the other involving the primarily inhibitory PD-1 receptor and its ligands, PD-L1 and PD-L2 have been identified. Hutloff A, Dittrich A M, Beier K C, Eljaschewitsch B. Kraft R, et al. 1999. ICOS is an inducible T-cell co-stimulator structurally and functionally related to CD28. Nature 397:263 66; Swallow M M, Wallin J J, Sha W C. 1999. B7h, a novel costimulatory homolog of B7.1 and B7.2, is induced by TNF. Immunity 11:423 32; Ishida Y, Agata Y, Shibahara K, Honjo T. 1992. Induced expression of PD-1, a novel member of the immunoglobulin gene superfamily, upon programmed cell death. EMBO J. 11:3887 95; Freeman G J, Long A J, Iwai Y, Bourque K, Chernova T, et al. 2000. Engagement of the PD-1 immunoinhibitory receptor by a novel B7 family member leads to negative regulation of lymphocyte activation. J. Exp. Med. 192:1027 34. Two additional inhibitory ligands, B7-H3 and B7-H4, and an inhibitory receptor, BTLA, also have been recently described. Chapoval A I, Ni J, Lau J S, Wilcox R A, Flies D B, et al. 2001. B7-H3: a costimulatory molecule for T cell activation and IFN-production. Nat. Immunol. 2:269 74; Sica G L, Choi I H, Zhu G F, Tamada K, Wang S D, et al. 2003. B7-H4, a molecule of the B7 family, negatively regulates T cell immunity. Immunity 18:849 61; Watanabe N, Gavrieli M, Sedy J R, Yang J F, Fallarino F, et al. 2003. BTLA is a lymphocyte inhibitory receptor with similarities to CTLA-4 and PD-1. Nat. Immunol. 4:670-79; Palucka A K, Laupeze B, Aspord C, Saito H, Jego G, Fay J, Paczesny S, Pascual V, Banchereau J. Immunotherapy via dendritic cells. Adv Exp Med Biol. 2005; 560:105-14. Researchers are still probing for receptors for B7-H3 and B7-H4 and also a possible additional receptor for PD-L1 indicating that there are additional pathways within the B7:CD28 superfamily to be characterized. The complexity of T cell signaling involving these known and unknown pathways suggests that therapeutic manipulation of multiple pathways may be necessary to induce an effective tolerance for the treatment of autoimmune and other immunological disorders, and to prevent transplant rejection.

Tolerogenic antigen presentation systems have been described extensively in the literature using live cells. One live cell approach is based on dendritic cells (DCs) with specific phenotypes. Rutella S, Lemoli R M. Regulatory T cells and tolerogenic dendritic cells: from basic biology to clinical applications. Immunol Lett. 2004 Jun. 15; 94(1-2):11-26; Akbar S M, Murakami H, Horiike N, Onji M. Dendritic cell-based therapies in the bench and the bedsides. Curr Drug Targets Inflamm Allergy. 2004 September; 3(3):305-10; Adorini L, Giarratana N, Penna G. Pharmacological induction of tolerogenic dendritic cells and regulatory T cells. Semin Immunol. 2004 April; 16(2):127-34. In vitro or in vivo generation of immature or semi-immature DCs have been considered as an important choice for inducing tolerance to autoantigens, transplants and for treating allergic conditions. Another approach involves modifying the surface of DCs and other conventional and non-conventional APCs so that tolerance can be achieved upon their interaction with T cells. This can achieved either by coating the surface of APCs with antibodies and ligands or by genetically expressing these molecules. It has been demonstrated that these approaches are useful in suppressing auto and allo-immune responses and preventing and/or treating the diseases. In one recent study, it was demonstrated that engagement of inhibitory receptor CTLA-4 on T cells from an antigen pulsed matured DC can not only induce significant tolerance against that antigen but also regulatory T cells (Tregs) with antigen specificity. This observation suggests that TCR ligation has to be simultaneous with the T cell negative regulator ligation for inducing antigen specific tolerance.

The receptor-ligand interactions that contribute to T cell activation or tolerance induction are complex and difficult to assess, being influenced by various parameters such as ligand densities, presence of coreceptors, receptor-ligand affinities and surface conditions. Hence, live cell based system for the induction of tolerance or immune response could be the best option in obtaining absolute physiological cell-cell interaction. However, live cell based systems can produce more significant deleterious effects. Live cell based systems are purely dependent on the regulated expression of surface molecules as in the case of immature of semi-mature dendritic cells. Minor changes in the conditions can influence the surface levels of various molecules and can produce adverse effect. Further, the cytokine and chemokine milieu in the recipient's body can have a significant effect on the maturation/activation state of the in vivo administered cells. This can lead to an unexpected outcome. Expression of molecules of interest exogenously may help overcome the problem to a certain extent, but it may be difficult to express several molecules exogenously at therapeutically relevant levels on the same cell. Generation of tolerance depending on their involvement in T cell functioning and differentiation. It may also be important that some stimulatory signal has to be combined with one or more dominant inhibitory signal to induce an effective antigen specific tolerance. Antigen specificity is well controlled at the epitope level and both CD4⁺ and CD8⁺ T cells can be targeted specifically. This approach can be used to prepare artificial antigen presenting system constructs not only using recombinant ligands but can be combined with anti-receptor antibodies and certain cytokines. Once prepared, these microspheres can be used in essentially the same ways as antigen or ligand bearing cells with respect to quantitation by counting, characterization of surface composition and density by flow cytometry and stimulation of lymphocyte signaling and functional responses. Although these artificial antigen-presenting systems can be generated for both stimulating and suppressing immune responses by changing the ligands, not many attempts have been made in vivo. Whereas, antigen coated or loaded biodegradable microspheres have been tested for enhanced immune response in several instances. What is needed is a biodegradable microsphere based tolerogenic based antigen presentation system wherein the system is used for the treatment of autoimmune conditions and other immune mediated disorders where T cells specific to antigenic epitopes are involved.

### BRIEF SUMMARY OF THE INVENTION

It is an object of the present invention to provide a biodegradable microsphere based antigen presenting system (mAPS). This system incorporates a recombinant MHC-peptide complex and one or more co-stimulatory/inhibitory ligands on the surface of cell sized biodegradable (injectable) microspheres (FIG. 1). These mAPS can be used either to induce tolerance (tmAPS) or to activate immune response (amAPS) based on the composition of co-ligands on the surface of the microsphere(s). They can also be used to specifically target either CD4⁺ or CD8⁺ effector cells specifically using MHC II and MHC I peptide complexes. This system 1) can be used to induce tolerance to any number of antigenic epitopes; 2) can be used to tolerize T cells against any type of autoimmune and other immune mediated disorders where at least one predominant antigenic epitope is identified; and 3) can be easily standardized to provide consistency that may be difficult with live cell based tolerance induction system. Furthermore, the passive nature of the artificial system allows for control over the number of molecules to be engaged. Any number of ligands, including cytokines such as TGF-β1 can be incorporated onto this mAPS. The herein described antigen specific tolerance strategy has an immense application in treating a wide spectrum of clinical conditions including both organ specific and systemic autoimmune conditions.

It is an object of the present invention to provide antigen specific tolerance, generated by using the presently described microsphere based antigen presenting system (mAPS). This system, for example, can engage antigen specific TCR and one or more T cell negative regulatory receptors simultaneously. The microspheres of this system are optionally biodegradable. “Microspheres” and “microsomes” are herein used interchangeably.

It is another object of the present invention to provide an artificial antigen presenting microsphere. The microsphere is biodegradable and may be composed of, for example, a peptide, a liposome, a non-liposome, sugar, protein carrier, and/or a polymer. The microspheres of the present invention may be coated with one or more MHC molecules and one or more T-cell receptor ligand, wherein the MHC molecules may, or may not be, presenting one or more antigens. The microspheres of the present invention may be modified with, for example, carboxyl groups (i.e. carboxylated), amines, sulf-hydryl groups, carbo-hydrates, any hydroxyl groups. One of ordinary skill in the art will recognize that cross-linking agents may be used to link various ligands, proteins, and peptides to the surface of a modified microsphere. Furthermore, the microspheres of the present invention may be modified to provide molecules and/or proteins which aid in ligand adherence; for example, protein A, biotin, avidin, extravidin, streptavidin, protein G, portein L, protein A/G, antibody, and anti-antibody. In an embodiment of the present invention, the microspheres carry ligands which may be immobilized onto the microsphere surface via chemicals such as glutaraldehyde, formaldehyde, and paraformaldehyde.

It is another object of the present invention to provide artificial biodegradable microspheres having capacities equivalent to a natural antigen presenting cell to bind to and induce an antigen-specific T-cell response. These artificial biodegradable microspheres may comprise combination of MHC molecules (optionally carrying or presenting one or more antigens) coated thereon, as well as other functional molecules including accessory molecules, co-stimulation molecules, adhesion molecules, and other immunomodulatory molecules such as cytokines.

In one embodiment, accessory molecules may be used to facilitate and stabilize the interaction between the antigen specific T cell and the MHC:antigen complex. In this embodiment, an example of an accessory molecule is LFA-1. Other accessory molecules include, but are not limited to CD11a/18, CD54(ICAM-1), CD106(VCAM), and CD49d/29(VLA-4), as well as antibodies to each of these molecule's ligands.

In another embodiment, the artificial biodegradable microspheres include inhibitory molecules that function to inhibit or repress an antigen-specific T cell. Suitable inhibitory molecules include B7.1wa, CTLA-4 binding proteins, PD-L1, HVEM.

In another embodiment, the artificial biodegradable microspheres include co-stimulatory molecules that function to stimulate or activate an antigen-specific T cell. One form of activation is cell proliferation. Suitable co-stimulatory molecules include, but are not limited to, B7.1, B7.2, CD5, CD9, CD2, CD40 and antibodies to their ligands. Preferably, such co-stimulatory molecules can be produced by recombinant methods. Co-stimulatory molecules can be used for a variety of purposes in addition to eliciting cell proliferation. For example, it is known that memory CD4+ T cells express B7-2 whereas naive CD4+ T cells do not. Neither type cell expresses B7-1 (Hakamada-Taguchi, R. European Journal of Immunology. 28:865-873.). Thus, the current invention may be used to selectively target memory T cells by incorporating anti-B7-2 into the artificial APC complex.

In another embodiment, the artificial antigen presenting microspheres also include adhesion molecules to facilitate strong and selective binding between the artificial APC and antigen-specific T cells. Suitable adhesion molecules include, but are not limited to, proteins of the ICAM family, for example ICAM-1 and ICAM-2, GlyCAM-1, as well as CD34, anti-LFA-1, anti-CD44 and anti-beta7 antibodies, chemokines, and chemokine receptors such as CXCR4 and CCR5, and antibodies to Selectins L, E, and P. Such molecules are known to be important as homing molecules for cells destined for specific locations in vivo. For example, α4β7 and L-selectin have been proposed as gut and peripheral lymph node homing molecules respectively. α4β7 is expressed mainly on memory T cells while L-selectin is expressed mainly on naive T cells (Abitorabi, M. A. J Immunol. 156:3111-3117.). It is also known that endothelial selectins (E-selectin and P-selectin) are associated with the extravasasion of T cells into inflammatory sites in the skin (Tietz, W. J. Immunol. 161:963-970.). In the current invention a β7 binding molecule and gut addressing MAdCAM-1, or an anti-L-selectin antibody may be incorporated into the artificial antigen presenting microspheres to distinguish further the type of T cell binding to the MHC:antigen complex.

In yet another embodiment, the artificial antigen presenting microspheres may be used in methods to induce antigen specific tolerance, wherein the method comprises contacting a T-cell with a biodegradable microsphere as described herein, wherein at least one MHC molecule and at least one T-cell receptor ligand are coated onto the microsphere. For inducing an antigen specific response, it is preferred that the T-cell receptor ligand is an inhibitory ligand. Such methods are useful, for example, in transplant rejections. In such cases, the MHC molecule can carry donor specific antigen or, alternatively, it may be free from carrying an antigen and act as an antigen itself (as a donor specific MHC molecule).

In yet another embodiment, the artificial antigen presenting microspheres may be used in methods for treating human recipients of tissue, cell and/or organ transplants from a human or non-human donor. Such methods comprise administering to the patient a pharmaceutical composition comprising an artificial antigen presenting microsphere, comprising (a) a biodegradable microsphere, (b) at least one MHC:donor specific antigen or donor specific MHC molecule coated onto the microsphere; and (c) at least one T-cell receptor ligand coated onto the microsphere, wherein the T-cell receptor ligand is an inhibitory ligand.

In yet another embodiment, the artificial antigen presenting microspheres may be used in methods for treating a patient suffering from an autoimmune disease, comprising: administering to the patient a pharmaceutical composition comprising an artificial antigen presenting microsphere, comprising (a) a biodegradable microsphere, (b) at least one MHC molecule coated onto the microsphere; and (c) at least one T-cell receptor ligand coated onto the microsphere, wherein the T-cell receptor ligand is an inhibitory ligand.

In yet another embodiment, the artificial antigen presenting microspheres of the present invention may be used in methods for treating patients suffering from one or more allergies. Such methods comprise, for example, administering to the patient a pharmaceutical composition comprising an artificial antigen presenting microsphere, comprising (a) a biodegradable microsphere, (b) at least one allergen antigen carrying MHC molecule coated onto the microsphere; and (c) at least one T-cell receptor ligand coated onto the microsphere, wherein the T-cell receptor ligand is an inhibitory ligand.

In any presently described immune tolerance inducing method which incorporates the use of the artificial antigen presenting microspheres of the present invention, the at least one T-cell receptor ligand may be, for example, a death inducing ligand whereby antigen specific T cells are killed, resulting in down regulation of the immune response. Such death inducing ligands include, but are not limited to, FAS-L, TRAIL, Anti-FAS agonistic antibody, and anti-TRAIL-agonistic antibody. These ligands will induce immune response suppression through inducing antigen specific T cell death and thereby reducing T cell number. The MHC molecules as used in the presently described methods and compositions may be useful in activating allo- or xeno-reactive T-cells.

In another embodiment, the artificial antigen presenting microspheres of the present invention may be used in methods for treating patients suffering from one or more infections, wherein the methods comprise, for example, administering to the patient a pharmaceutical composition comprising an artificial antigen presenting microsphere, comprising (a) a biodegradable microsphere, (b) at least one protective self-antigen molecule carrying MHC molecule (MHC:protective self-antigen molecule) coated onto the microsphere; and (c) at least one T-cell receptor ligand coated onto the microsphere, wherein the T-cell receptor ligand is an activating ligand (a positive regulator or upregulator of T-cell response). Such a method is useful for expanding protective antigen specific T cells, suppressor T cells and regulatory T cells in the patient's body.

In another embodiment, the artificial antigen presenting microspheres of the present invention may be used in methods for treating patients suffering from one or more infections, wherein the methods comprise, for example, administering to the patient a pharmaceutical composition comprising an artificial antigen presenting microsphere, comprising (a) a biodegradable microsphere, (b) at least one protective self-antigen molecule carrying MHC molecule (MHC:protective self-antigen molecule) coated onto the microsphere; and (c) at least two T-cell receptor ligands coated onto the microsphere, wherein one T-cell receptor ligand is an activating ligand (a positive regulator or upregulator of T-cell response) and one other T-cell receptor ligand is an inhibitory ligand (down regulator of T-cell response). Such a method is useful for expanding protective antigen specific T cells, suppressor T cells and regulatory T cells in the patient's body.

In yet another embodiment of the present invention, the artificial antigen presenting microspheres may be used in methods for treating infections. Such methods comprise administering to a patient in need thereof a pharmaceutical composition comprising an artificial antigen presenting microsphere, comprising (a) a biodegradable microsphere, (b) at least one foreign antigen carrying MHC molecule coated onto the microsphere; and (c) at least one T-cell receptor ligand coated onto the microsphere, wherein the T-cell receptor ligand is an activating ligand (upregulator of T-cell response).

In yet another aspect of the present invention, the artificial antigen presenting microspheres may be used in methods for expanding protective antigen specific T cells, suppressor T cells and/or regulatory T cells. These expanded T-cell populations may then be used for treating a patient. Such a method comprises, for example, obtaining T cells from a patient, culturing or incubating the T cells with a composition comprising an artificial antigen presenting microsphere of the present invention (for example, comprising (a) a microsphere, (b) at least one MHC, or MHC:antigen, molecule coated onto the microsphere; and (c) at least one T-cell receptor ligand coated onto the microsphere, wherein the T-cell receptor ligand is an inhibitory or activating ligand), expanding the T cells, and administering a pharmaceutical composition comprising the expanded T-cell population to the patient. Furthermore, or optionally, the T-cells can be incubated/cultured with a composition comprising an artificial antigen presenting microsphere of the present invention (for example, comprising (a) a microsphere, (b) at least one MHC, or MHC:antigen, molecule coated onto the microsphere; and (c) at least one T-cell receptor ligand coated onto the microsphere, wherein the T-cell receptor ligand is an inhibitory or activating ligand) in the presence of one or more soluble factors. These soluble factors will aid in the differentiation or conversion of the T cells to be expanded. As used herein, differentiation and conversion are used interchangeably. Differentiation or conversion relates to the induction of changes in the functional and phenotypic characteristics of the T-cell. For example, a T cell can have no specific known role prior to differentiation or conversion; however, after the conversion or differentiation, it can have a specific role or property. A T cell can function as a self-destructive (auto-reactive) T cell prior to conversion; however, it can function as a self-protective cell after conversion. A T cell can have no specific influence on another T cell before conversion; however, it can indirectly (via the secretion of cytokine, for example) or directly (via contact) suppress or activate another T cell after conversion. Furthermore, a T cell may be able to recognize a bacterial cell, a virus, or a tumor cell but may not be able to eliminate the infection. Upon conversion, the T cell may be able to clear or suppress the infection.

T-cells undergoing differentiation often secrete factors which can disrupt the differentiation process of T-cells. The one or more soluble factors, which can include antibodies, can bind to these secreted factors and inhibit or prevent them from exerting their function on T-cells. Other one or more soluble factors that can aid in the differentiation of T-cells include, but are not limited to, cytokines and chemokines.

Once one or more T-cells have been differentiated or converted to the cell type of interest, the converted one or more T-cells can be expanded by inducing proliferation, for example, for the purpose of obtaining sufficient number of cells for treatment. One can obtain a small number of T-cells from the patient, differentiate and/or expand the obtained population in vitro, and administer a larger number of cells to the patient as treatment.

The above-described artificial antigen presenting microsphere compositions and methods are particularly useful in therapeutic applications relating to autoimmune diseases, such as autoimmune gastritis, diabetes, autoimmune hemolytic anemia, autoimmune neutropenia, pernicious anemia, polyarteritis nodosa systemic lupus erythematosus, Wegener's granulomatosis, Autoimmune hepatitis, Behçet's disease, Crohri's disease, Primary bilary cirrhosis, Scleroderma, Ulcerative colitis, Sjögren's syndrome, Type 1 diabetes mellitus, Uveitis, Graves' disease, Thyroiditis, Type 1 diabetes mellitus, Myocarditis, Rheumatic fever, Scleroderma, Ankylosing spondylitis, Rheumatoid arthritis, Glomerulonephritis, Systemic lupus erythematosus, Type 1 diabetes mellitus, Rheumatoid arthritis, Sarcoidosis, Scleroderma, Systemic lupus erythematosus, Dermatomyositis, Myasthenia gravis, Polymyositis, Guillain-Barré syndrome, Multiple sclerosis, Systemic lupus erythematosus, Alopecia greata, Pemphigus/pemphigoid, Psoriasis, Scleroderma, Systemic lupus erythematosus, and Vitiligo.

In vitro methods are also contemplated herein, such that the herein described microspheres are optionally non-biodegradable. Examples of materials for producing a non-biodegradable antigen presenting microsphere include, but are not limited to, polystyrene, magnetic, paramagnetic, and latex.

### BRIEF DESCRIPTION OF THE DRAWINGS

FIG. 1. MHC-peptide antigen complex A (a) and MHC-peptide antigen complex B(b) and negative regulatory receptor binding proteins/ligands along with positive regulatory receptor binding protein (activator ligand), or MHC-peptide antigen complex A (c) and MHC-peptide antigen complex B(d) and negative regulatory receptor binding proteins/ligands alone coated microspheres when cultured in vitro with antigen specific T cells or injected into mice having antigen specific T cells (autoreactive or allo-reactive) (e,f) interact with T cells with respective antigen specificities (g) and suppress T cells and induce antigen specific tolerant T cells (h). Co-stimulatory (positive regulatory/activator) ligand is not needed for an effective antigen specific tolerance induction, but helps improve the magnitude of tolerance. Other positive regulatory/activator ligands and antibodies can also be used along with the negative regulatory ligands and antibodies to induce an effective T cell tolerance and antigen specific tolerant or suppressor/regulatory T cells.

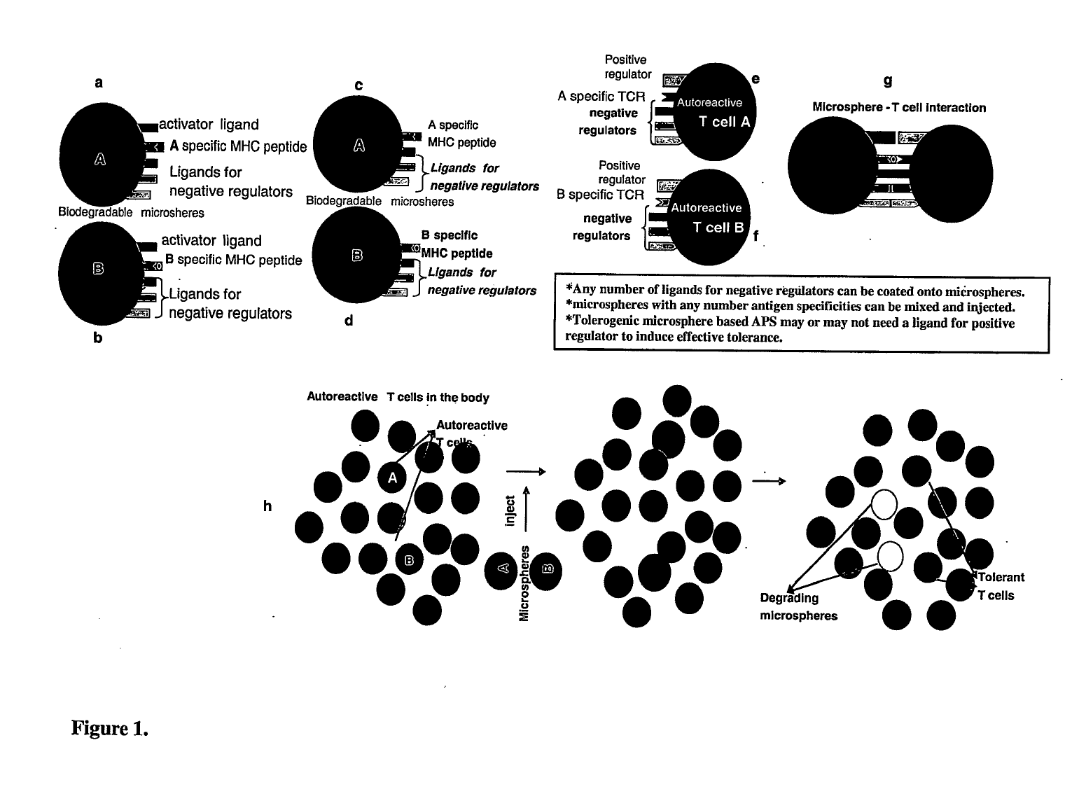

FIG. 2. (15) Regulation of T cell activation by microsphere bound negative regulators: NOD.BDC2.5 TCR transgenic (A) and D011.10 TCR transgenic (B) mice (Balb/c mice) were primed with specific peptides (ISQAVHAAHAEINEAGR—SEQ ID NO:1) and YVRPLWVRME (SEQ ID NO:2) peptide respectively intravenously along with LPS. Mice were sacrificed 3 days after injection and spleen cells were collected, CD4+ T cells were isolated by negative selection and cultured along with microspheres carrying different MHCs and ligands as mentioned below each panel. Cells that were cultured without microspheres and with blank (non-coated) microspheres are the extreme end panels. Cells were collected at 24 h culture and stained with FITC labeled CD69 and PE-labeled CD4 antibodies and tested by FACS for activation marker. I-Ag7 and I-Ad are MHC class molecules of NOD and Balb/c mice. Recombinant I-Ad-peptide contains the peptide sequence ISQAVHAAHAEINEAGR (SEQ ID NO: 1), and I-Ag7-peptide contains the peptide sequence YVRPLWYRME (SEQ ID NO:2) as antigens. These results indicate that artificial antigen presenting system can suppress antigen specific T cell activation when negative regulatory ligands are used in the presence or absence of costimulatory molecules. Further, MHC molecules with peptide sequence linked at cDNA level or empty MHC molecules passively loaded or covalently linked with antigenic peptides can be used in the biodegradable artificial antigen presenting system.

FIG. 3. (16) MHC-peptide antigen complex A (a) and MHC-peptide antigen complex B(b) and negative regulatory receptor binding proteins coated microspheres when cultured in vitro with antigen specific T cells or injected into mice having antigen specific T cells (autoreactive or allo-reactive) (c,d) interact with T cells with respective antigen specificities (e,f) and suppress T cells and induce antigen specific tolerent suppressor T cells (g). Costimulator (positive regulatory) ligand may or may not be needed for an effective antigen specific tolerance induction. Any number of MHC-peptide combinations, negative and positive regulatory ligands can be coated onto the microsphere.

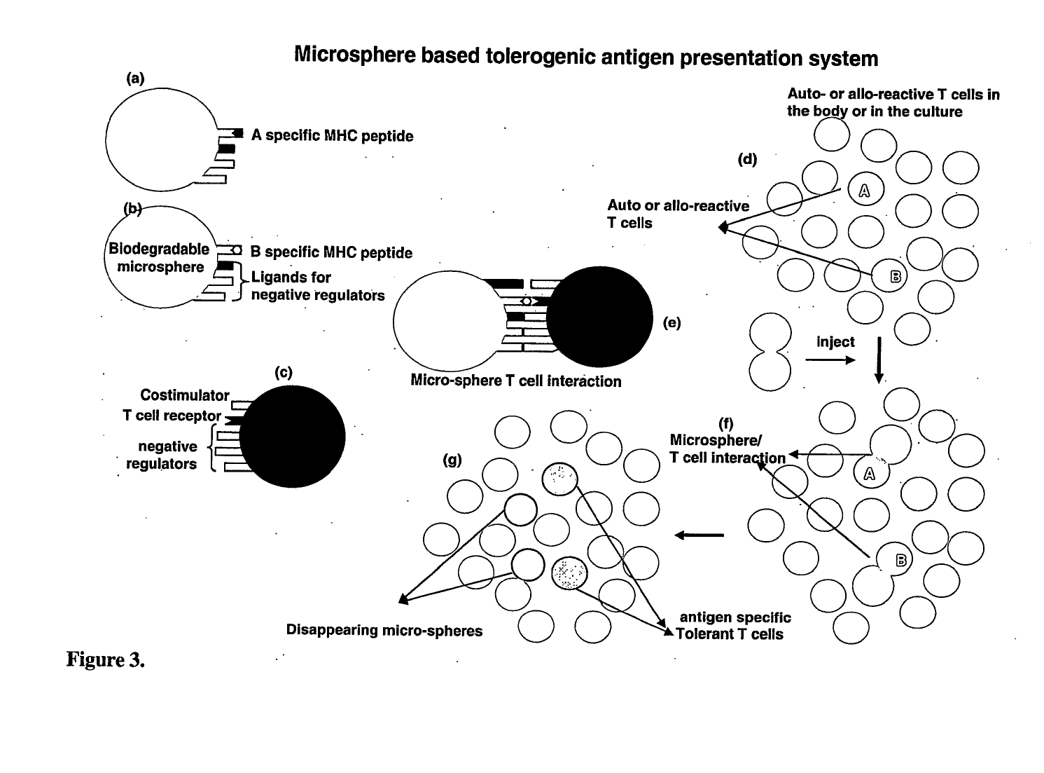

FIG. 4. (19) Recombinant MHC and costimulatory (B7.2) molecules on biodegradable microspheres can activate antigen specific T cells: Recombinant empty or antigenic peptide carrying MHC (I-Ad and I-Ag7, specific for Balb/c DO11.10 and NOD BDC2.5 mice respectively) and B7.2 molecules were coated onto biodegradable microspheres, cultured with CFSE labeled T cells from DO11.10 and BDC2.5 TCR-transgenic mice for 5 days, stained with anti-CD4 antibody linked to fluorochrome PE, and tested for divided (proliferated) CD4+ T cells by FACS method. Results show that MHC molecule carrying antigenic peptide, but not empty MHC molecules, bind to antigen specific T cells and induce activation and proliferation. These results also show that recombinant MHC molecules carrying antigenic peptide can be used to develop artificial antigen presenting system/cells to induce T cell activation or for inducing T cell tolerance if used along with a right combination of negative regulatory ligands, antibodies and other molecules. Other T cell stimulatory molecules and CD28 antibody can be used along with recombinant MHC molecules carrying peptides on artificial antigen presenting cells.

FIG. 5. Recombinant MHC, co-stimulatory and co-inhibitory molecules on biodegradable microspheres can suppress antigen specific T cell activation and proliferation depending on the combinations of molecules used. Recombinant empty or antigenic peptide carrying MHC (I-Ad and I-Ag7, specific for Balb/c DO11.10 and NOD BDC2.5 mice respectively) and B7.2, and negative regulatory molecules were coated onto biodegradable microspheres, cultured with CFSE labeled T cells from DO11.10 and BDC2.5 TCR-transgenic mice for 5 days, stained with anti-CD4 antibody linked to fluorochrome PE, and tested for divided (proliferated) CD4+ T cells by FACS method. Results show that MHC molecules carrying antigenic peptides, but not empty MHC molecules, bind to antigen specific T cells and induce activation and proliferation. Use of costimulator B7.2 along with MHC-peptide can induce activation and proliferation of T cells. However; addition of negative regulatory ligand B7.1wa alone or in combination with PD-L1, HVEM, and B7-H4 leads to suppression of T cell proliferation significantly compared to antigen presenting system carrying only MHC-peptide and B7.2 molecules. These results also show that recombinant MHC molecules carrying antigenic peptide can be used to develop artificial antigen presenting system/cells to induce T cell activation as well as for inducing T cell tolerance if used along with a right combination of negative regulatory ligands, antibodies and other molecules. Moreover, different negative ligand combinations can suppress T cell proliferation to different levels. A complete suppression of T cell proliferation is not necessary for these T cells to become suppressor or regulatory T cells.

FIG. 6. Recombinant MHC, co-stimulatory and co-inhibitory molecules on biodegradable microspheres can induce suppressor/regulatory T cells. Recombinant empty or antigenic peptide carrying MHC (I-Ag7, specific for NOD BDC2.5 mice), B7.2 and other negative regulatory molecules were coated onto biodegradable microspheres, cultured with T cells from BDC2.5 TCR-transgenic mice for 5 days. These T cells were then restimulated using BDC2.5 peptide pulsed dendritic cells as antigen presenting cells to check if they can respond to further stimulation with the antigen. Culture supernatants were collected from these cultures and tested for immunosuppressive cytokines IL-10 and TGF-b1. Results show that T cells cultured in the presence of microspheres coated with negative ligands become IL-10 and TGF-beta1 producing suppressor T cells. Use of costimulator B7.2 along with MHC-peptide that induced activation and proliferation of T cells did not induce suppressor T cells. These results also show that different negative ligand combinations can induce different types of suppressor T cells and T cell tolerance. Therefore, negative ligands can be used individually or in combinations along with MHC molecule carrying antigenic peptide to induce antigen specific tolerance and suppressor/regulatory T cells.

FIG. 7. Recombinant MHC, costimulatory (B7.2) and negative regulatory molecule coated biodegradable microspheres (artificial antigen presenting cells/system) can reach immune and other target organs to exert tolerogenic effect. Poly-lactate based red fluorescent biodegradable microspheres were coated with recombinant I-Ag7 dimer (MHC molecule), B7.2, B7.1wa, PD-L1 and HVEM molecules. These labeled microspheres where injected intravenously into NOD mice (10×106 beads/mouse), mice were sacrificed after 24 hours, spleen, lymph node, pancreas and liver tissues were collected, 7 micrometer cryo-sections were made and observed under a fluorescence microscope. Bright red spots represent microspheres in the tissue. Left panel for each sample represents phase contrast view of a specific tissue section. These results show that artificial antigen presenting cells/system can reach organs where immune response occurs, and induce the desired effect of immune modulation (either T cell suppression and regulatory T cell induction or activation depending on the molecules present on the surface of microspheres). As expected, spleen (a major site for immune response) receive maximum number of microspheres. Microsphere based antigen presenting system can reach any tissue or organ to which blood circulation occurs. Cell size of these microspheres may also make them enter lymphatic circulation and induce antigen specific immune suppression. Since biodegradable microspheres are made up of biological materials, they can disintegrate and disappear from the body without causing any side effect after inducing desired immune modulation.

FIG. 8. Treatment of type 1 diabetes using biodegradable artificial antigen presenting system. IAg7-cDNA vector constructs with mouse Ig tag were modified to link different immunodominant peptides (IDPs), expressed in S2 Drosophila cells in serum free medium, purified using protein A columns. These peptides are known self antigens specific to pancreatic beta cells. Empty MHC or individual types of MHC-peptides (I-Ag7) were coated onto biodegradable microspheres along with indicated ligands, injected intravenously to NOD mice at the age of 8 weeks or 12 weeks (10×106 beads/mouse; 10 mice/group). These mice were tested for blood glucose levels every week as an indication of hyperglycemia associated with type 1 diabetes. For I-Ag7-peptide group, 2×106 microspheres carrying a different antigenic peptide (5 different antigenic peptides separately), along with indicated ligands, we're mixed to obtain 10×106 beads/mouse. These results show that self antigen specific peptide carrying MHC molecules and negative regulatory ligands together on artificial antigen presenting cell/system can suppress self reactive T cells and prevent type 1 diabetes. Also, since a single injection with the artificial antigen presenting system could delay the disease dramatically, multiple injections will be useful to treat already established diabetes. This system can be used to treat any autoimmune disease and transplant rejection just by changing MHC-peptides.

FIG. 9. Negative regulator ligand mediated suppression of T cell response. CD4+ T cells isolated from D011.10 TCR-transgenic mice were incubated with Ova peptide pulsed BMDC that were coated either with isotype control antibodies, anti-CTLA-4 Ab or a mixture of anti-CTLA-4, anti-PDL-1 Abs and recombinant HVEM-Ig. T cells from these cultures were analyzed for activation markers CD69 and CD25 after 24 h (a and b). Percentage positive cells in CD4+ gated population is shown in the inner rectangle. CFSE stained T cells were tested for the proliferation pattern on day 5 (c). Spent medium collected from these cultures after 48 hr were tested for cytokines using luminex multiplex assay (d). Cells from similar cultures were stained with specific antibodies for Treg markers and analyzed by FACS(e). These results show that engagement of individual or multiple negative regulatory receptors on T cells using ligand combinations along with antigen presentation through MHC can induce antigen specific regulatory/suppressor T cells that may or may not produce suppressor cytokines. These regulatory T cells are tolerant to antigen stimulation and can suppress other T cells with the same antigen specificity. These results support the use of negative regulators and other co-stimulatory molecules individually or in combinations on biodegradable artificial antigen presenting system/cells for immune modulation and for treating clinical conditions.

FIG. 10. Blockade of B7.2 induces TGF-b1 expressing regulatory/suppressor T cells: Ova pulsed DCs were cocultured with ova primed T cells (both total and CD4+CD25+ depleted populations) in the presence of various antibodies for 7 days. These cells were washed, and plated for another 24 hr in fresh medium and tested for surface expression of CD25 and TGF-b1 and intracellular expression of FoxP3 on CD4+ cells by FACS. Live CD4+ cells were gated (using anti-CD4 antibody -PE-TR and 7-AAD) for the graphs shown above. These results show that selective engagement of CTLA-4 by its preferential ligand B7.1 can induce antigen specific TGF-beta 1 producing suppressor/regulatory T cells. Therefore, ligands for CTLA-4 used in the artificial antigen presenting system along with MHC-peptide can produce antigen specific regulatory T cells both in vitro or in vivo.

### DETAILED DESCRIPTION OF THE INVENTION

The present invention is directed to novel compositions and methods for expanding and/or modulating T-cells. Additionally, the present invention is directed to methods of treating conditions that would benefit from the modulation of T cell responses, for example, transplantation therapies, autoimmune disorders, allergies, cancers and viral infections, and virtually any T-cell mediated disease. Furthermore, the present, invention is directed to novel artificial antigen presenting microspheres and methods incorporating the use of these microspheres for the in vitro expansion of T-cells.

The herein described system provides, for example, for the active suppression of T-cells. MHC molecules carrying antigen peptide present antigen to T-cells concurrently with negative regular ligands. These negative regulator ligands bind to negative regulatory receptors on T-cells so that T cells are actively suppressed against the specific antigen (and tolerance to that specific antigen achieved). The combination of negative regulators, individually or at varying combinations, along with MHC molecules, alone or carrying antigen peptide, direct the induction of antigen specific tolerance. The regulators and MHC molecules are coated onto the presently described microspheres. Any number and types of MHC molecules, plus or minus antigen, and regulator TCR binding proteins can be combined to obtain the desired effect of antigen specific tolerance. The present system can be used to treat or prevent, for example, autoimmune diseases and transplant rejection where at least one antigen or epitope is identified. The herein described microspheres, preferably biodegradable or biocompatible, can be administered, for example injected, in vivo to suppress antigen specific T cells and/or to induce antigen specific T cell suppression and suppressor/regulatory T cells to prevent or treat a specific condition. These MHC and negative regulator ligand coated microspheres can also be used in vitro to generate antigen specific suppressor/regulatory T cells by culturing with T cells from the patients. The resultant T cells can be injected back to the patient to treat the target disease.

The term “coat” refers to immobilizing the ligands and MHC molecules of the present invention onto the surface of individual microspheres. This may be accomplished via well known methods in the art; for example, covalent linkage, chemical treatment (glutaraldehyde, etc.), etc.

The microsphere may be biodegradable or non-biodegradable. Biodegradable microspheres contemplated by the present invention include, for example, a peptide, a liposome, a non-liposome, sugar, protein carrier, and/or a polymer. The microspheres of the present invention may be coated with one or more MHC molecules and one or more T-cell receptor ligands. The MHC molecules may, or may not, be carrying or presenting one or more antigen(s).

According to the preferred embodiments of the present invention, an artificial antigen presenting microsome is presented which may or may not be biodegradable. Examples of biodegradable microsomes include, but are not limited to, biodegradable polymers such as polylactide, poly(lactic acid-co-glycolic acid), poly(dioxanone), poly(trimethylene carbonate) copolymer; poly(caprolactone) homopolymer, polyanhydride, polyorthoester, polyphosphazene, poly(caprolactone) copolymer, any polymeric substances based on polylactide (PLA), polyglycolide (PGA), polycaprolactone (PCL), Poly(ethylene oxide), Poly-alginate, PEO, Poly((lactide-co-ethyleneglycol)-co-ethyloxyphosphate), Poly(LAEG-EOP), Poly(1,4-bis(hydroxyethyl)terephthalate-co-ethyloxyphosphate), Poly(BHET-EOP), Poly(1,4-bis(hydroxyethyl)terephthalate-alt-ethyloxyphosphate)-co-1,4-bis(hydroxyethyl)terephthalate-co-terephthalate), Poly(BHET-EOP/TC, 80/20), and PMMA (Polymethylmethacrylate). Other biodegradable core components of the presently described microspheres include, but are not limited to, modified poly(saccharide)s, e.g., starch, cellulose, and chitosan; proteins (e.g., collagen, albumin, gelatin, elastin, silk fibroin), lipid microspheres (e.g., prepared using lecithin and vegetable oils; and beta-estradiol microsphere). Liposomes are also contemplated by the present invention, wherein they can be modified to display the ligand on their surface. For example, liposomes carrying protein A, Protein L, protein G, Protein A/G, streptavidin, avidin, extravidin, biotin, antibodies, can be used to coat ligands on their surface.

The microsomes of the present invention may comprise MHC:antigen complexes, accessory molecules and other functional molecules including, but not limited to, co-stimulatory molecules, adhesion molecules, modulation molecules, inhibitory molecules, irrelevant molecules, and labels which are collectively coated onto the microsome.

Accessory molecules may be included for the purpose of stabilizing the interaction between a T-cell receptor (TCR) and an MHC or MHC:antigen complex. Suitable accessory molecules may include, but are not limited to, LFA-1, CD49d/29(VLA-4), CD 11a/18, CD54(ICAM-1), and CD106(VCAM) and antibodies to their ligands.

Co-stimulatory molecules may be included for the purpose of stimulating or activating a TCR. Suitable co-stimulatory molecules may include, but are not limited to, B7-1, B7-2, CD5, CD9, CD40, ICOS-L, Ox40-L, IL-2, IL-7, IFN-gamma, IL-12, IL-15, IL-17, IL-18, IL-22, TNF-α, LFA-3, ICAM-1, anti-CD28 agonistic antibody, anti-CTLA-4 antagonistic antibody, anti-ICOS agonistic antibody, anti-PDL1-antagonistic antibody, anti-PDL-2 antagonistic antibody, anti-B7-H3-receptor antagonistic antibody, and anti-B7-H4 receptor antagonistic antibody.

Inhibitory molecules may be included for the purpose of down regulating a T-cell response via interaction with a TCR. Suitable inhibitory molecules include, but are not limited to B7.1wa, CTLA-4 binding proteins, PD-L1, HVEM, PDL-2, B7-H3, B7-H4, OX-2, TGF-beta1, IL-10, IL-4, natural and recombinant anti-CTLA-4 agonistic antibody, anti-PD-1 agonistic antibody, Anti-B7-H3 receptor agonistic antibody, Anti-B7-H4 receptor antibody, anti-CD28 antagonistic antibody, and anti-ICOS antagonistic antibody.

Irrelevant molecules may be included for the purpose of either carrying a label or serving as a scaffold for binding to a solid support. Such a molecule can be any peptide or other molecule having characteristics that make it suitable for use with a liposome and antigen carrier. Such molecule should not interfere with the binding of a T cell to the artificial antigen presenting microsome.

The MHC molecules coated onto the microsome of the present invention include, but are not limited to, MHC I (HLA-type I):antigen, MHC II (HLA-type II):antigen, MHC I (HLA-type I) itself as an antigen, MHC II (HLA-type II) itself as an antigen.

With respect to the MHC:antigen complexes coated onto microsomes, antigens include, but are not limited to, self antigens (e.g. antigenic peptides, of insulin, insulin, GAD, GAD65, HSP, thyroglobulin, nuclear proteins, acetylcholine receptor, collagen, TSHR, ICA512(IA-2) and IA-2β (phogrin), carboxypeptidase H, ICA69, ICA12, thyroid peroxidase), peptides, histocompatibility allo- and xeno-antigens, and peptides of allergenic proteins. Furthermore, such antigens may be selected from the group consisting of a peptide derived from the recipient for graft versus host diseases, a cancer cell-derived peptide, a donor derived peptide, a pathogen-derived molecule, a peptide derived by epitope mapping, a self-derived molecule, a self-derived molecule that has sequence identity with the pathogen-derived antigen, the sequence identity having a range selected from the group consisting of between 5 and 100%, 15 and 100%, 35 and 100%, and 50 and 100%.

The aforementioned molecules of interest may be produced by recombinant technology as is well known to those skilled in the art. Use of recombinantly produced molecules further provides the opportunity to produce such molecules as chimeras or fusion molecules.

In still another embodiment, the artificial antigen presenting microsomes of the present invention comprise labels wherein a label is associated with at least one of a group selected from the group consisting of a lipid bilayer of a liposome, a lipid of a liposome, a polylactide, poly(lactic acid-co-glycolic acid), poly(dioxanone), poly(trimethylene carbonate) copolymer, poly(caprolactone) homopolymer, polyanhydride, polyorthoester, polyphosphazene, poly(caprolactone) copolymer, any polymeric substances based on polylactide (PLA), polyglycolide (PGA), polycaprolactone (PCL), Poly(ethylene oxide), Poly-alginate, PEO, Poly((lactide-co-ethyleneglycol)-co-ethyloxyphosphate), Poly(LAEG-EOP), Poly(1,4-bis(hydroxyethyl)terephthalate-co-ethyloxyphosphate), Poly(BHET-EOP), Poly(1,4-bis(hydroxyethyl)terephthalate-alt-ethyloxyphosphate)-co-1,4-bis(hydroxyethyl)terephthalate-co-terephthalate), Poly(BHET-EOP/TC, 80/20), PMMA (Polymethylmethacrylate), modified poly(saccharide)s, e.g., starch, cellulose, and chitosan; proteins (e.g., collagen, albumin, gelatin, elastin, silk fibroin), lipid microspheres (e.g., prepared using lecithin and vegetable oils; and beta-estradiol microsphere), an antigen, an MHC molecule, a co-stimulatory molecule, an inhibitory molecule, a cell modulation molecule, an irrelevant molecule, and an accessory molecule.

In another aspect of the present invention, a method of isolating T cells specific for an antigen of interest is presented. Such a method comprises, for example, (a) obtaining a biological sample containing T cells which are specific for an antigen of interest; (b) preparing an artificial antigen presenting microsome as described herein; (c) contacting the biological sample obtained in step (a) with the artificial antigen presenting microsome obtained in step (b) to form an artificial antigen presenting microsome:T cell complex; (d) removing the complex formed in step (c) from the biological sample; and (e) separating T cells specific for the antigen of interest from the complex. Any suitable biological sample which contains T cells specific for the antigen of interest may be used in the method. Suitable biological samples containing T cells specific for an antigen of interest include fluid biological samples, such as blood, plasma and cerebrospinal fluid, and solid biological samples, such as tissue, for example, histological samples.

In one embodiment, the artificial antigen presenting microsome may be complexed to a solid support in addition to the T cell. The complexing of the artificial antigen presenting microsome to the solid support provides a means to anchor the artificial antigen presenting microsome so that it and any T cell binding to it can be preferentially captured and isolated from extraneous matter. In such case, the solid support may be a glass or magnetic bead that is coated with, for example, a lipid mono layer that is bound to the bead by, for example, a linker. The solid support may additionally have noncovalently bound accessory molecules associated with the lipid monolayer such as binding molecules that recognize and bind to irrelevant molecules associated with the artificial microsome. In another embodiment, the binding molecules may be covalently bound to the solid support by a linker.

Methods for inducing the conversion or differentiation of T-cells are also contemplated by the present invention. For example, a method for inducing differentiation of T cells, wherein the method comprises obtaining T cells from a patient, culturing or incubating the T cells with a composition comprising the artificial antigen presenting microsphere described herein, and converting to a differentiated T cell. Examples of differentiated T cells include, but are not limited to, suppressor T cells, regulatory T cells, effector T cells, specific cytokine producing T cells, and cytotoxic T cells.

The microspheres disclosed herein may have a size of between about 0.2 μm and 50 μm for administration with a needle. For needle-injected microspheres, the microsphere may have any appropriate dimensions so long as the longest dimension of the microsphere permits the microsphere to move through a needle. This is generally not a problem in the administration of microspheres. In a preferred embodiment, the microsphere is between about 2 μm and 20 μm in diameter. In another preferred embodiment, the microsphere is 5 μm in diameter. In yet another preferred embodiment, the microsphere size can be as low as 0.2 μm and as high as 500 μm for in vitro use; for example, in cell culture systems for expanding/generating suppressor regulatory T cells for therapy or for inducing T-cell differentiation.

The above-described artificial antigen presenting microsphere compositions and methods are particularly useful in therapeutic applications relating to autoimmune diseases, such as autoimmune gastritis, diabetes, autoimmune hemolytic anemia, autoimmune neutropenia, pernicious anemia, polyarteritis nodosa systemic lupus erythematosus, Wegener's granulomatosis, Autoimmune hepatitis, Behçet's disease, Crohn's disease, Primary bilary cirrhosis, Scleroderma, Ulcerative colitis, Sjögren's syndrome, Type 1 diabetes mellitus, Uveitis, Graves' disease, Thyroiditis, Type 1 diabetes mellitus, Myocarditis, Rheumatic fever, Scleroderma, Ankylosing spondylitis, Rheumatoid arthritis, Glomerulonephritis, Systemic lupus erythematosus, Type 1 diabetes mellitus, Rheumatoid arthritis, Sarcoidosis, Scleroderma, Systemic lupus erythematosus, Dermatomyositis, Myasthenia gravis, Polymyositis, Guillain-Barré syndrome, Multiple sclerosis, Systemic lupus erythematosus, Alopecia greata, Pemphigus/pemphigoid, Psoriasis, Scleroderma, Systemic lupus erythematosus, and Vitiligo.

The compounds utilized in the compositions and methods of this invention can also be modified by appending appropriate functionalities to enhance selective biological properties. Such modifications are known in the art and include those which increase biological penetration into a given biological system (e.g., blood, lymphatic system, central nervous system), increase oral availability, increase solubility to allow administration by injection, alter metabolism and alter rate of excretion.

According to a preferred embodiment, the compositions of this invention are formulated for pharmaceutical administration to a mammal, preferably a human being.

Such pharmaceutical compositions of the present invention can be administered orally, parenterally, by inhalation spray, nasally, or via an implanted reservoir. The term “parenteral” as used herein includes subcutaneous, intravenous, intramuscular, intra-articular, intra-synovial, intrasternal, intrathecal, intrahepatic, intralesional and intracranial injection and infusion techniques. Preferably, the compositions are administered orally, intravenously, or nasally.

Sterile injectable forms of the compositions of this invention can be aqueous or oleaginous suspension. These suspensions can be formulated according to techniques known in the art using suitable dispersing or wetting agents and suspending agents. The sterile injectable preparation can also be a sterile injectable solution or suspension in a non-toxic parenterally acceptable diluent or solvent. Among the acceptable vehicles and solvents that can be employed are water, Ringer's solution, phosphate buffer saline (PBS), and isotonic sodium chloride solution. In addition, sterile, fixed oils are conventionally employed as a solvent or suspending medium. Fatty acids, such as oleic acid and its glyceride derivatives are useful in the preparation of injectables, as are natural pharmaceutically-acceptable oils, such as olive oil and castor oil, especially in their polyoxyethylated versions. These oil solutions or suspensions can also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms can also be used for the purposes of formulation.

If a solid carrier is used, the preparation can be tableted, placed in a hard gelating capsule in powder or pellet form, or in the form of a troche or lozenge. The amount of solid carrier will vary, e.g., from about 25 mg to 400 mg. When a liquid carrier is used, the preparation can be, e.g., in the form of a syrup, emulsion, soft gelatin capsule, sterile injectable liquid such as an ampule or nonaqueous liquid suspension. Where the composition is in the form of a capsule, any routine encapsulation is suitable, for example, using the aforementioned carriers in a hard gelatin capsule shell.

A syrup formulation can consist of a suspension or solution of the compound in a liquid carrier for example, ethanol, glycerine, or water with a flavoring or coloring agent. An aerosol preparation can consist of a solution or suspension of the compound in a liquid carrier such as water, ethanol or glycerine; whereas in a powder dry aerosol, the preparation can include e.g., a wetting agent.

Formulations of the present invention comprise an active ingredient together with one or more acceptable carrier(s) thereof and optionally any other therapeutic ingredient(s). The carrier(s) should be “acceptable” in the sense of being compatible with the other ingredients of the formulation and not deleterious to the recipient thereof.

The pharmaceutical compositions of this invention can be orally administered in any orally acceptable dosage form including, but not limited to, capsules, tablets, and aqueous suspensions or solutions. In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents can also be added.

It will be recognized by one of skill in the art that the form and character of the pharmaceutically acceptable carrier or diluent is dictated by the amount of active ingredient with which it is to be combined, the route of administration, and other well-known variables.

Upon improvement of a patient's condition, a maintenance dose of a compound, composition or combination of this invention can be administered, if necessary. Subsequently, the dosage or frequency of administration, or both, can be reduced, as a function of the symptoms, to a level at which the improved condition is retained when the symptoms have been alleviated to the desired level, treatment should cease. Patients can, however, require intermittent treatment on a long-term basis upon any recurrence or disease symptoms.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of active ingredients will also depend upon the particular compound and other therapeutic agent, if present, in the composition.

Although the present invention has been described with reference to preferred embodiments, persons skilled in the art will recognize that changes may be made in form and detail without departing from the spirit and scope of the invention. A role for B7.1, but not B7.2, C-domain in CTLA-4 engagement and down-regulation of xeno-response has been identified. The C-domain of B7:1 (but not that of B7.2) plays significant role in the high affinity interactions with CTLA-4. This property of the C-domain provides B7.1 with a potential to inhibit T cell function even when the B7.2 is expressed at higher level. Since we have identified the T cell down modulatory role of B7.1, we speculate that B7.1 may have a role in inducing regulatory T cells (Tregs). Targeted ligation of CTLA-4 can induce target specific tolerance. In one of the studies, it is shown that engagement of CTLA-4 on thyroid specific autoreactive T cells by thyrocyte bound anti-CTLA-4 prevents experimental autoimmune thyroiditis (EAT) in a murine model. Very interestingly, the disease suppression was mediated by TGF-β1 producing antigen specific CD4⁺CD25⁺ regulatory T cells generated as a result of targeted CTLA-4 engagement. In another study, ligation of CTLA-4 from allogenic target by the target bound anti-CTLA-4 antibody can induce allo-antigen specific tolerance though CD4+CD25+CTLA-4^{high} Treg induction and with the help of TGF-beta1 and IL-10.

DC based therapeutic strategies for autoimmune diseases have also been designed. A subset of tolerogenic DCs that capable of inducing IL-10 producing CD4+CD25+ Tregs, with the potential to suppress Tg specific T cell response both in vitro and in vivo, was expanded in these mice by treating with low dose GM-CSF. These tolerogenic DCs could not only prevent EAT, but also suppress ongoing disease even at late stage significantly. Tregs induced by these DCs could suppress autoimmune response upon adoptive transfer and this Treg function could be blocked using anti-IL10R antibody. This GM-CSF induced tolerogenic DCs had no effect on the outcome of an antibody mediated autoimmune Grave's disease.

### Example 1

### Differential Role of B7.1 and B7.2 in T Cell Tolerance

Bone marrow derived DCs or DCs purified from spleens were pulsed with ovalbumin (Ova) and maturation was induced using LPS for 24 hr and cultured in the presence of T cells from naïve or Ova primed mice and B7.1, B7.2 and anti-CTLA-4 blocking antibodies. Interestingly, T cells from Ova primed mice, but not naïve mice, showed significantly lowered T cell activation and proliferation, IL-2 and IFN-γ responses, but an increased IL-4 and IL-10 production in the presence of anti-B7.2 antibody compared to isotype control or B7.1 antibody. Although T cells from these cultures containing anti-B7.2 antibody showed no increase in CD4⁺CD25⁺ T cells compared to controls, interestingly, a significant number of CD4⁺ T cells from this culture showed increased TGF-β1 surface expression. Tertiary stimulation of these T cells induced much stronger IL-4 and IL-10 responses, but undetectable level of secreted TGF-β1. Co-culture of these T cells with CFSE stained ova primed T cells showed that T cells from B7.2 blocking experiments could suppress Ova specific CD4 and CD8 T cell responses suggesting their negative regulatory properties. Studies are underway—using B7.1 and B7.2 knockout mice, D011.10 TCR transgenic and NOD-BCD2.5 TCR transgenic mice to understand the mechanism and if this is just a mouse strain dependent effect. Although the above results were obtained using Balb/c mice, we confirmed a similar effect upon B7.2 blocking in C57BL/6 and CBA/J strains. Our results using DO11.10 TCR transgenic mice and NOD-BCD2.5 TCR transgenic mice demonstrated that blocking B7.2 using anti-B7.2 antibody could suppress T cell response significantly.

### Example 2

### Induction of Immune Tolerance and Tregs Using DC Directed CTLA-4 Ligation

A novel approach was designed to generate robust antigen specific tolerance and Tregs. In this approach, antigen pulsed mature DCs that were coated with cross-linking anti-CTLA-4 antibody were used to induce tolerance and Tregs to that specific antigen. DCs were pulsed with either ovalbumin and coated with anti-CTLA-4 antibody and injected intravenously into mice that had been primed with this antigen. Mice administered with anti-CTLA-4 coated mature DC, but not immature DC, produced antigen specific T cell suppression suggesting that surface bound antibodies are rapidly internalized by immature DC and not available for interacting with CTLA-4. Mice injected with anti-CTLA-4 antibody coated mature DC showed significantly suppressed T cell proliferation and IL-2 production but increased IL-10 and TGF-β1 response upon ex vivo restimulation with the same antigen compared to mice that received DCs coated with isotype control antibody. These mice showed a significant increase in CD4⁺CD25⁺ Treg cell population. Upon ex vivo stimulation with Ova, T cells from mice that received anti-CTLA-4 ab coated DCs showed low early activation marker (CD69) and also fewer cells entering the memory cell compartment (CD62L^{low}) compared to controls (not shown). Most interestingly, when the mice (test group) were immunized with ova and treated twice with anti-CTLA-4 ab coated Ova pulsed DCs and rested for 15 days, the memory CD4⁺ T cell numbers were lower compared to the control group. However, these mice showed about 70% more CD4⁺CD25⁺ cells. While the naïve CD25⁺ T cell (CD4⁺CD25⁺CD62L^{high}) numbers were more or less same in test and control groups, memory Tregs (CD4⁺CD25⁺CD62L^{low}) numbers were about 150% more in test mice compared to control mice. Intracellular staining for Foxp3 (using Foxp³ detection kit, Ebiosciences) of these cells revealed that CD4+CD25+CD62L^{low} cells from anti-CTLA-4 ab coated DC received mice, but not from the control mice, are Foxp3 positive. These Tregs could suppress Ova specific T cell response more significantly compared to CD4⁺CD25⁺ Tregs with naïve phenotype. This suggests that a large number of the memory cells generated in the test group have Treg phenotype. Similar observation was made in EAT model using thyroglobulin and the effect of Treg induction in preventing and treating thyroiditis is being studied (Data not shown).

### Example 3

### DC Directed CTLA-4 Engagement Study in NOD Mice

The DC directed CTLA-4 engagement approach in NOD mice was tested in vitro. A pool of three GAD65 peptides (GAD206-226, GAD217-236 and GAD286-300), that are the primary and some of the earliest targets for autoreactive T cells in NOD mice^{(135,136)}, were used as antigen in an in vitro study using T cells collected from diabetic mice. Though the numbers were small, we observed that autoreactive T cell proliferation to these peptides suggesting that T cells specific to these peptides are present in diabetic mice. DCs collected from pre-diabetic mice were pulsed with these peptides, induced maturation, coated with anti-CTLA-4 or control Ab and tested against T cells from diabetic mice. Anti-CTLA-4 ab coated DCs suppressed T cell response significantly compared to control Ab coated DCs when T-cells from Diabetic mice and naïve DCs-were used. Cells from these cultures were collected on day 7, washed, rested for 3 days, and analyzed for CD4⁺CD25⁺ Tregs by FACS. The number of CD4⁺ cells expressing CD25 in T cells that were incubated with anti-CTLA-4 coated DCs was higher compared to controls and these T cells could suppress effector function of T cells from diabetic mice. Encouraged by the above in vitro results, we carried out a experiments in vivo to test the efficacy of DC directed CTLA-4 engagement in inducing Tregs and suppressing autoimmune response in NOD mice. Randomly picked fourteen weeks old NOD mice were intravenously injected with 2×10⁶ anti-CTLA-4 antibody or control antibody coated DCs and monitored for glucose levels every week. 5 out of 6 control Ab coated DC received mice showed glucose levels above 200 mg/dl within 5 week after DC treatment. Whereas, only 2 out 6 mice that received anti-CTLA-4 ab coated DCs showed glucose level above 200 mg/dl. Moreover, these mice demonstrated a slow increase (relative to control mice), or stabilization of glucose levels. This result suggests that anti-CTLA-4 ab coated DCs may have induced Tregs in vivo and these T cells may be responsible for the suppression of autoimmunity.

Tregs can be generated by different means and antigen specificity can be added by co-ligation of antigen specific TCR and tolerognic receptor. The phenotypes and nature of inhibition induced by Tregs generated under different conditions differently may also be different. For example, CD4+CD25+ regulatory T cell populations with three different cytokine induction patterns (TGF-beta1 alone, TGF-beta1, IL-4 and IL10 or IL-10 alone) have been observed in three different studies. It is interesting to note that CTLA-4 engagement alone could induce different type of Tregs in EAT and in the allo-response models. This suggests that the type of TCR engagement and microenvironment can also play significant role in the type of Tregs generated. The studies presented herein, strengthen the notion that functionally and/or phenotypically different sub-populations of CD4⁺CD25⁺Tregs exist. The Treg inducing property of CTLA-4 mediated negative signaling alone or in combination with the other T cell negative regulators such as PD-1 and BTLA can be exploited in a microsphere based tolerogenic antigen presenting system for developing a more flexible, reliable and simple therapeutic strategy for autoimmune diseases.

### Example 4

### Biodegradable Microsphere Based Strategy In Vitro

Studies were undertaken to develop microsphere based tolerogenic antigen presenting system (mAPS). Biodegradable carboxylated-microspheres of 2 and 20 μm sizes were obtained from Kisker GBR, Germany and a more biologically relevant 5 μm size carboxylated regular and fluorescent microspheres are being custom synthesized. These particles are made from polylactide (PLA) with a density of 1.02 (suitable for in vivo use). They are stable at neutral pH and degradation starts through basic or acid pH or enzymatic hydrolysis. 2 μm microsphere were used in in vitro studies. Microspheres were activated using EDC-NHS (N-(3-dimethylaminopropyl)-N′-ethylcarbodiimide hydrochloride (EDC) and N-hydroxysuccinimide) method. Activated sterile microspheres were resuspended in PBS, specific antibodies or recombinant proteins were coated covalently and used in the assay. Microsphere antigen presenting systems (mAPSs) were made by coating these activated microspheres with MHC II molecules and co-stimulatory/inhibitory ligands and cross-linking antibodies. I-A^{d} was purified from M12 B cell lymphoma using anti-I-A^{d} antibody (HB-26, ATCC) by following previously described method⁽¹³⁵⁾. Microsphere bound I-A^{d} was passively loaded with Ova₍₃₂₃₋₃₃₉₎⁽¹³⁵⁾ before using in the assay. Recombinant I-A^{d} carrying this ova peptide is currently being produced using the constructs provided by Dr. Ton Schumacher, Leiden University Medical Center Netherlands. NOD mouse specific recombinant I-A^{g7} linked to BDC2.5 TCR transgenic CD4⁺ reactive peptide (YVRPLVVVRME—SEQ ID NO:2) or empty I-Ag⁷ (constructs were kindly provided by Dr. Jeffrey Bluestone, Diabetes Center, University of California) were purified from Drosophila S2 cells stably transfected with the above constructs as described earlier⁽¹³⁶⁾. Purified empty or peptide loaded MHC II molecules were mixed with relevant iigand(s)/antibody(ies) or normal mouse IgG (to equalize the total protein level) and incubated with activated microspheres, remaining binding sites were blocked using glycine and used in in vitro assays against CD4+ T cells from DO11.10 (Balb/c, I-A^{d} background) and BDC2.5 (NOD, I-A^{g7} background) TCR-transgenic mice. See Example 15.

### Example 5

### Recombinant MHC Molecules Bind to Antigen Specific T Cells

Recombinant empty or antigenic peptide carrying MHC molecules (I-Ad and I-Ag7, specific for Balb/c DO11.10 and NOD BDC2.5 mice respectively) were incubated with T cells from DO11.10 and BDC2.5 TCR-transgenic mice, further incubated with secondary antibodies and anti-CD4 antibody linked to fluorochromes and tested for binding on CD4+ T cells by FACS method. Results show that MHC molecule carrying antigenic peptide, but not empty MHC molecules, bind to antigen specific T cells. These results also show that recombinant MHC molecules carrying antigenic peptide can be used to develop artificial antigen presenting system/cells.

### Example 6

### Recombinant MHC, Costimulatory (B7.2) and Negative Regulatory Molecules can be Coated onto Biodegradable Microspheres to Prepare Artificial Antigen Presenting Cells/System

Poly-lactate based biodegradable microspheres were coated with mouse IgG (A), recombinant I-Ad dimer (MHC molecule) (B), a mixture of I-Ad dimer and recombinant B7.2 (C), or a mixture of I-Ad dimer, B7.2, B7.1wa, PD-L1, HVEM and B7-H4. Molecules bound on the surface of microspheres were detected using fluorochrome labeled specific antibodies to individual ligands or proteins by FACS method. These results show that multiple types of molecules can be coated onto biodegradable microspheres at different ratios to use as artificial antigen presenting cells/systems to induce or inhibit immune response using appropriate ligand and MHC combinations. Further, these ligands can be coated individually or in combinations onto any type of biodegradable microsphere prepared using sugars, proteins, lipids and/or other biological materials using any linking method.

### Example 7

### Recombinant MHC and Co-Stimulatory (B7.2) Molecules on Biodegradable Microspheres can Activate Antigen Specific T Cells

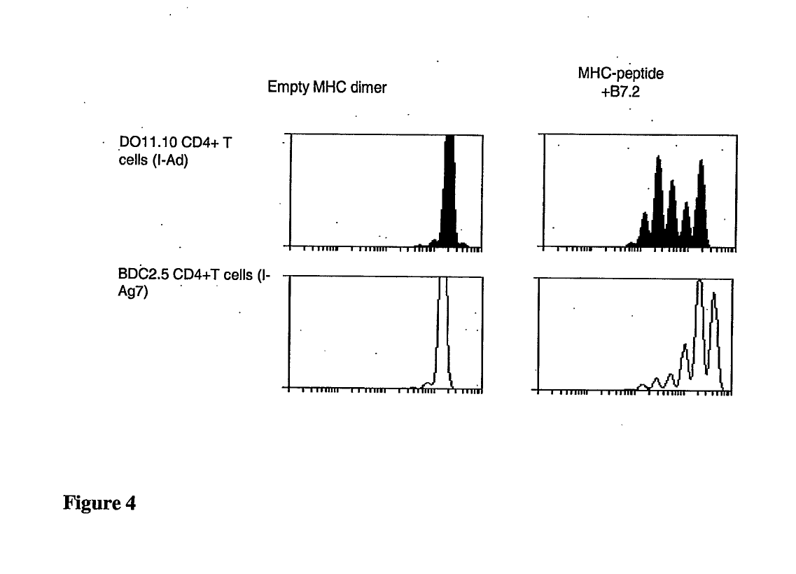

Recombinant empty or antigenic peptide carrying MHC (I-Ad and I-Ag7, specific for Balb/c DO11.10 and NOD BDC2.5 mice respectively) and B7.2 molecules were coated onto biodegradable microspheres, cultured with CFSE labeled T cells from DO11.10 and BDC2.5 TCR-transgenic mice for 5 days, stained with anti-CD4 antibody linked to fluorochrome PE, and tested for divided (proliferated) CD4+ T cells by FACS method. See FIG. 4. Results show that MHC molecule carrying antigenic peptide, but not empty MHC molecules, bind to antigen specific T cells and induce activation and proliferation. These results also show that recombinant MHC molecules carrying antigenic peptide can be used to develop artificial antigen presenting system/cells to induce T cell activation or for inducing T cell tolerance if used along with a right combination of negative regulatory ligands, antibodies and other molecules. Other T cell stimulatory molecules and CD28 antibody can be used along with recombinant MHC molecules carrying peptides on artificial antigen presenting cells.

### Example 8

### Recombinant MHC, Co-Stimulatory and Co-Inhibitory Molecules on Biodegradable Microspheres can Suppress Antigen Specific T Cell Activation and Proliferation Depending on the Combinations of Molecules Used

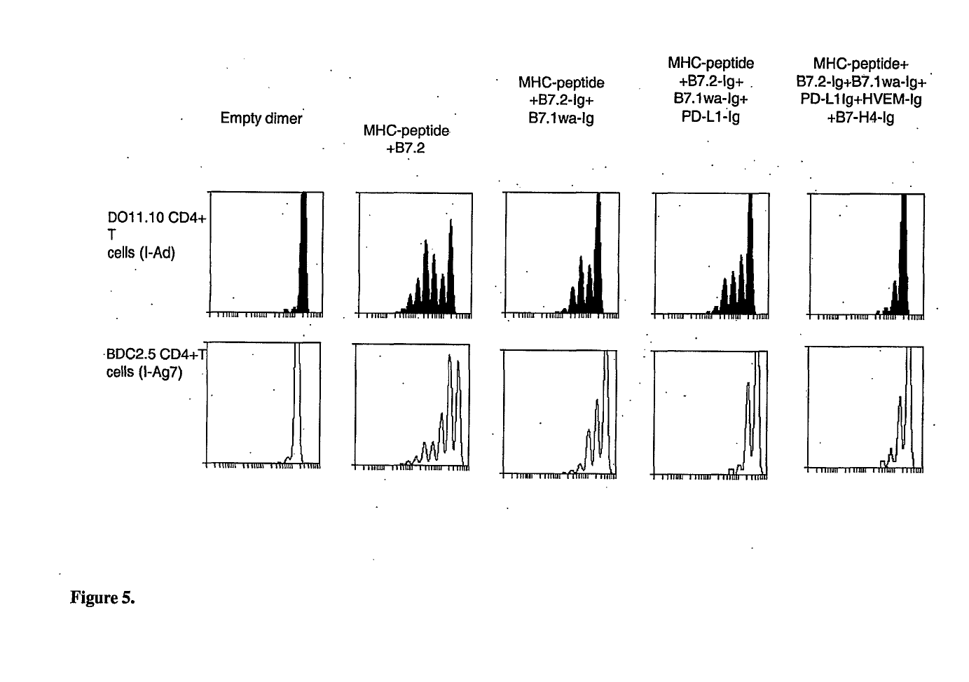

Recombinant empty or antigenic peptide carrying MHC (I-Ad and I-Ag7, specific for Balb/c DO11.10 and NOD BDC2.5 mice respectively) and B7.2, and negative regulatory molecules were coated onto biodegradable microspheres, cultured with CFSE labeled T cells from DO11.10 and BDC2.5 TCR-transgenic mice for 5 days, stained with anti-CD4 antibody linked to fluorochrome PE, and tested for divided (proliferated) CD4+ T cells by FACS method. See FIG. 5. Results show that MHC molecules carrying antigenic peptides, but not empty MHC molecules, bind to antigen specific T cells and induce activation and proliferation. Use of costimulator B7.2 along with MHC-peptide can induce activation and proliferation of T cells. However, addition of negative regulatory ligand B7.1wa alone or in combination with PD-L1, HVEM, and B7-H4 leads to suppression of T cell proliferation significantly compared to antigen presenting system carrying only MHC-peptide and B7.2 molecules. These results also show that recombinant MHC molecules carrying antigenic peptide can be used to develop artificial antigen presenting system/cells to induce T cell activation as well as for inducing T cell tolerance if used along with a right combination of negative regulatory ligands, antibodies and other molecules. Moreover, different negative ligand combinations can suppress T cell proliferation to different levels. A complete suppression of T cell proliferation is not necessary for these T cells to become suppressor or regulatory T cells.

### Example 9

### Recombinant MHC, Co-Stimulatory and Co-Inhibitory Molecules on Biodegradable Microspheres Can Induce Suppressor/Regulatory T Cells

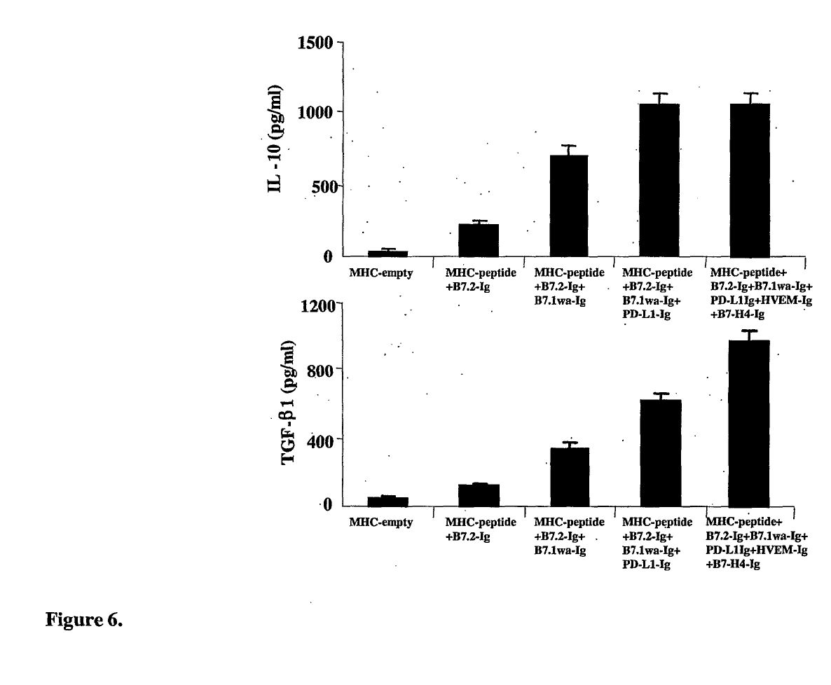

Recombinant empty or antigenic peptide carrying MHC (I-Ag7, specific for NOD BDC2.5 mice), B7.2 and other negative regulatory molecules were coated onto biodegradable microspheres, cultured with T cells from BDC2.5 TCR-transgenic mice for 5 days. These T cells were then restimulated using BDC2.5 peptide pulsed dendritic cells as antigen presenting cells to check if they can respond to further stimulation with the antigen. Culture supernatants were collected from these cultures and tested for immunosuppressive cytokines IL-10 and TGF-b1. Results show that T cells cultured in the presence of microspheres coated with negative ligands become IL-10 and TGF-beta1 producing suppressor T cells. See FIG. 6. Use of costimulator B7.2 along with MHC-peptide that induced activation and proliferation of T cells did not induce suppressor T cells. These results also show that different negative ligand combinations can induce different types of suppressor T cells and T cell tolerance. See FIG. 6. Therefore, negative ligands can be used individually or in combinations along with MHC molecule carrying antigenic peptide to induce antigen specific tolerance and suppressor/regulatory T cells.

### Example 10

### Recombinant MHC, Costimulatory (B7.2) and Negative Regulatory Molecule Coated Biodegradable Microspheres (Artificial Antigen Presenting Cells/System) can Reach Immune and Other Target Organs to Exert Tolerogenic Effect

Poly-lactate based red fluorescent biodegradable microspheres were coated with recombinant I-Ag7 dimer (MHC molecule), B7.2, B7.1wa, PD-L1 and HVEM molecules.

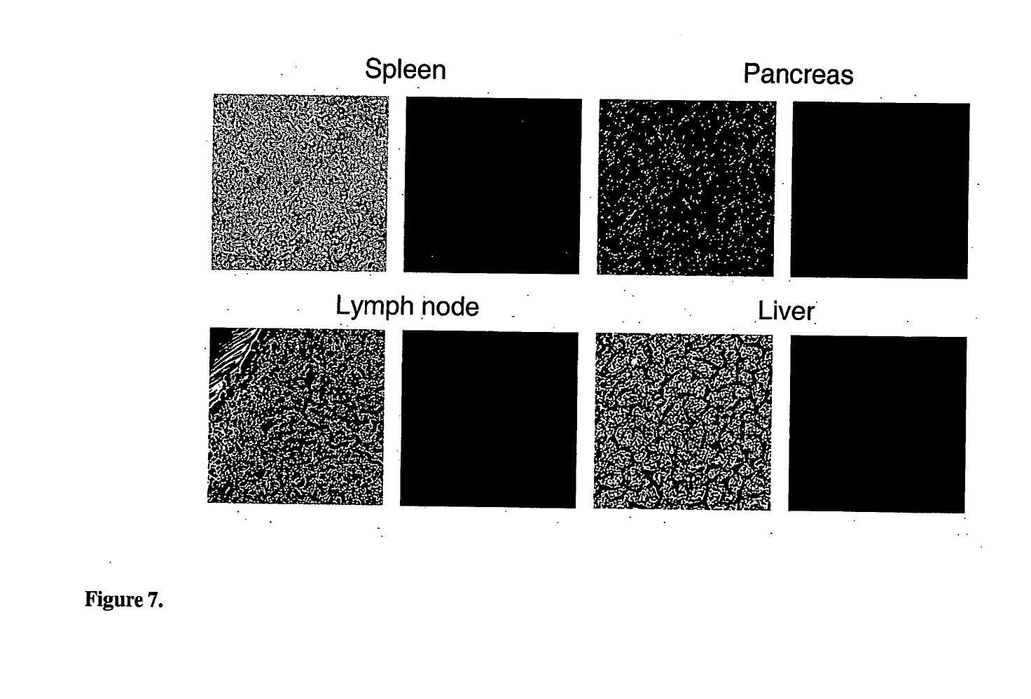

These labeled microspheres where injected intravenously into NOD mice (10×10⁶ beads/mouse), mice were sacrificed after 24 hours, spleen, lymph node, pancreas and liver tissues were collected, 7 micrometer cryo-sections were made and observed under a fluorescence microscope. See FIG. 7. Bright red spots represent microspheres in the tissue. Left panel for each sample represents phase contrast view of a specific tissue section. These results show that artificial antigen presenting cells/system can reach organs where immune response occurs, and induce the desired effect of immune modulation (either T cell suppression and regulatory T cell induction or activation depending on the molecules present on the surface of microspheres). As expected, spleen (a major site for immune response) receive maximum number of microspheres. See FIG. 7. Microsphere based antigen presenting system can reach any tissue or organ to which blood circulation occurs. Cell size of these microspheres may also make them enter lymphatic circulation and induce antigen specific immune suppression. Since biodegradable microspheres are made up of biological materials, they can disintegrate and disappear from the body without causing any side effect after inducing desired immune modulation.

### Example 11

### Recombinant dimeric MHC II (IAg7, NOD Mouse specific)-Peptide Panel

IAg7-cDNA vector constructs with mouse Ig tag were modified to link different immunodominant peptides (IDPs) mentioned below each graph, expressed in S2 Drosophila cells in serum free medium, purified using protein A columns. These peptides are some of the known self antigens specific to pancreatic beta cells.

NOD mice were immunized s.c. in CFA with equal amounts (5 ug each/mouse), rested for 15 days, lymph node cells were collected and antigen specific T cells were detected using purified MHC II dimers and alexa-flour 488 labeled protein A and tested in cells gated for CD4+ population by FACS.

These results show that a specific peptide linked MHC II molecules or peptide loaded MHC I or II molecule can bind only to a small number peptide antigen specific T cells. Therefore, artificial antigen presenting system/cells made using a specific MHC-peptide will selectively modulate T cells specific to that antigen, but not other T cells.

This property makes this artificial antigen presenting system ideal for targeted therapy without affecting the global immune response.

### Example 12

### Treatment of Type 1 Diabetes Using Biodegradable Artificial Antigen Presenting System

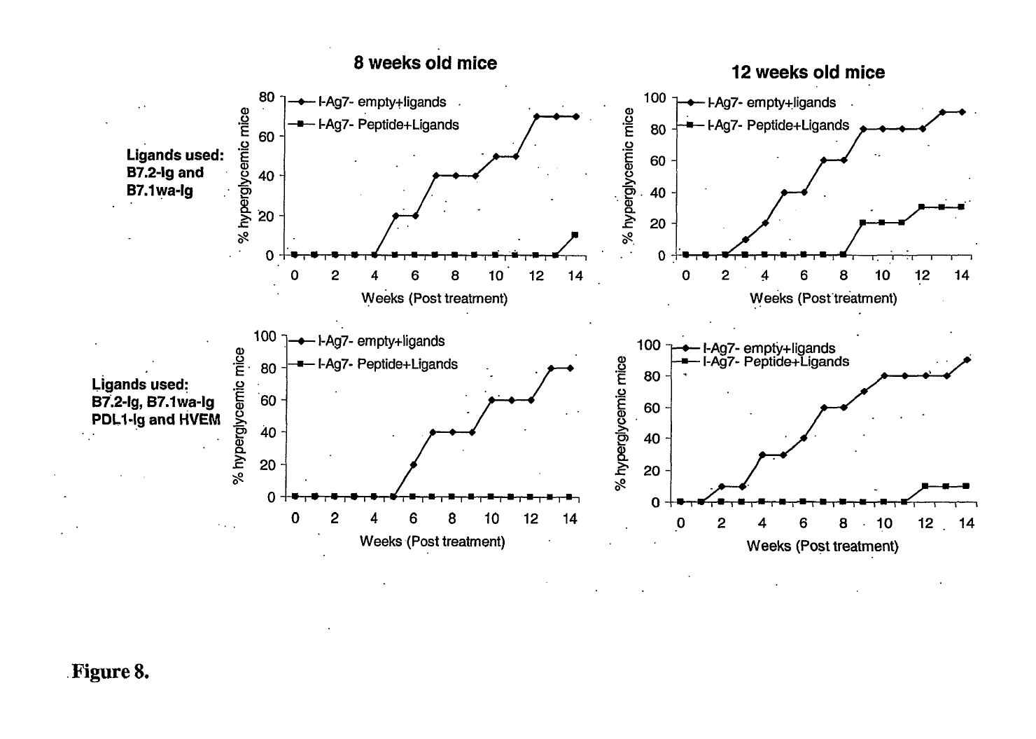

IAg7-cDNA vector constructs with mouse Ig tag were modified to link different immunodominant peptides (IDPs), expressed in S2 drosophila cells in serum free medium, purified using protein A columns. These peptides are known self antigens specific to pancreatic beta cells. Empty MHC or individual types of MHC-peptides (I-Ag7) were coated onto biodegradable microspheres along with indicated ligands, injected intravenously to NOD mice at the age of 8 weeks or 12 weeks (10×10⁶ beads/mouse; 10 mice/group). These mice were tested for blood glucose levels every week as an indication of hyperglycemia associated with type 1 diabetes. See FIG. 8. For I-Ag7-peptide group, 2×10⁶ microspheres carrying a different antigenic peptide (5 different antigenic peptides separately), along with indicated ligands, were mixed to obtain 10×10⁶ beads/mouse. These results show that self antigen specific peptide carrying MHC molecules and negative regulatory ligands together on artificial antigen presenting cell/system can suppress self reactive T cells and prevent type 1 diabetes. Also, since a single injection with the artificial antigen presenting system could delay the disease dramatically, multiple injections will be useful to treat already established diabetes. This system can be used to treat any autoimmune disease and transplant rejection just by changing MHC-peptides.

### Example 13

### Negative Regulator Ligand Mediated Suppression of T Cell Response

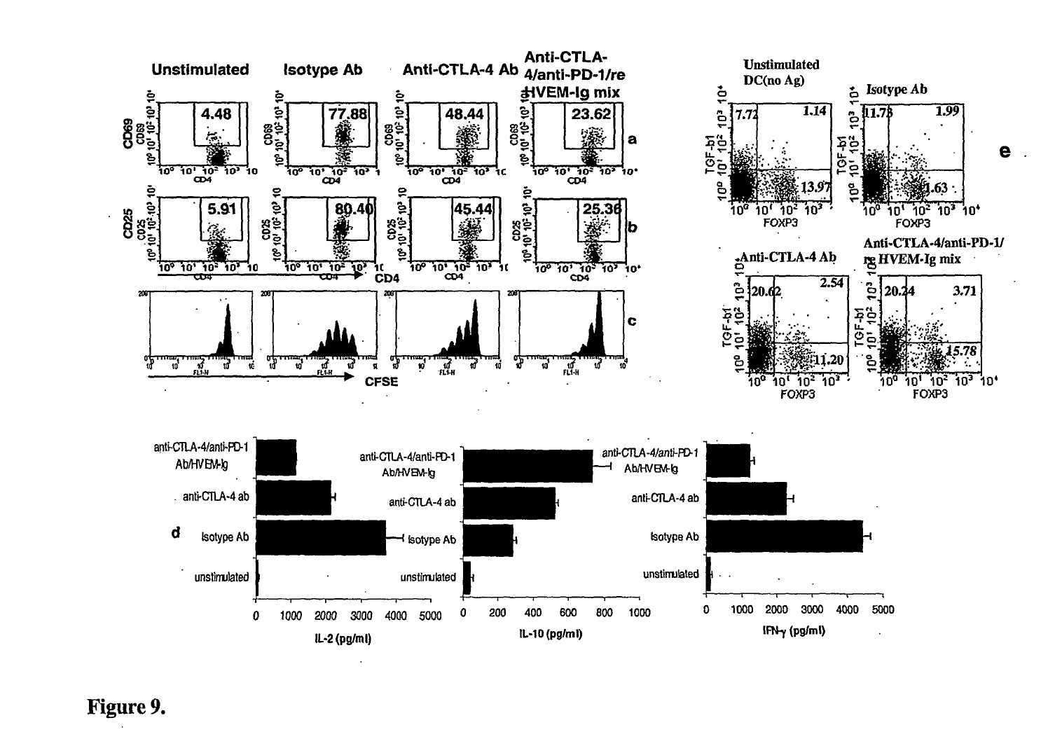

See FIG. 9. CD4+ T cells isolated from D011.10 TCR-transgenic mice were incubated with Ova peptide pulsed BMDC that were coated either with isotype control antibodies, anti-CTLA-4 Ab or a mixture of anti-CTLA-4, anti-PDL-1 Abs and recombinant HVEM-Ig. T cells from these cultures were analyzed for activation markers CD69 and CD25 after 24 h (a and b). Percentage positive cells in CD4+ gated population is shown in the inner rectangle. CFSE stained T cells were tested for the proliferation pattern on day 5 (c). Spent medium collected from these cultures after 48 hr were tested for cytokines using luminex multiplex assay (d). Cells from similar cultures were stained with specific antibodies for Treg markers and analyzed by FACS (e). These results show that engagement of individual or multiple negative regulatory receptors on T cells using ligand combinations along with antigen presentation through MHC can induce antigen specific regulatory/suppressor T cells that may or may not produce suppressor cytokines. These regulatory T cells are tolerant to antigen stimulation and can suppress other T cells with the same antigen specificity. These results support the use of negative regulators and other co-stimulatory molecules individually or in combinations on biodegradable artificial antigen presenting system/cells for immune modulation and for treating clinical conditions.

### Example 14

### Blockade of B7.2 Induces TGF-b1 Expressing Regulatory/Suppressor T Cells

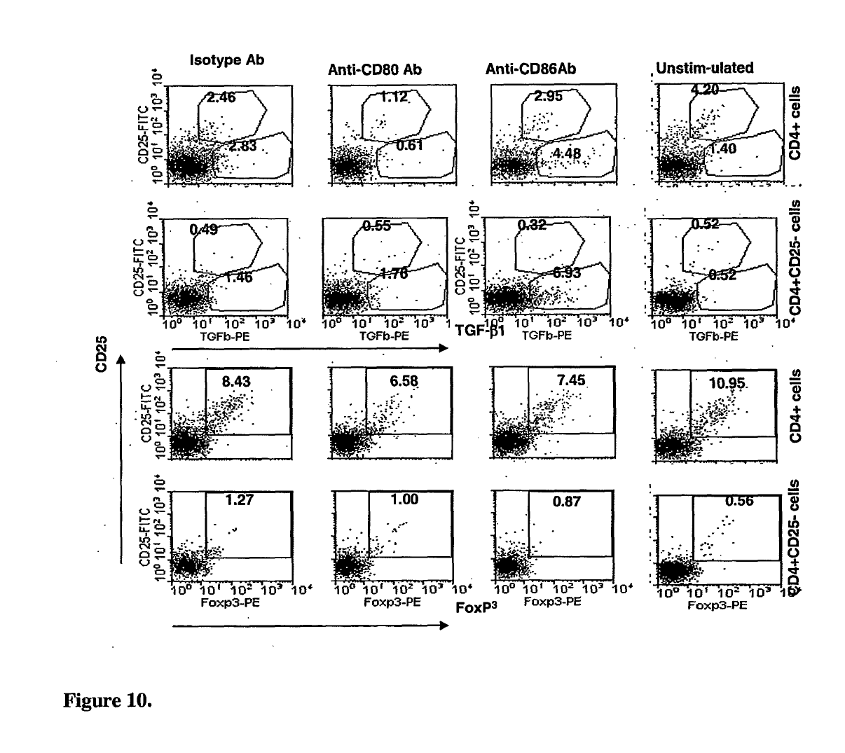

Ova pulsed DCs were co-cultured with ova primed T cells (both total and CD4+CD25+ depleted populations) in the presence of various antibodies for 7 days. These cells were washed, and plated for another 24 hr in fresh medium and tested for surface expression of CD25 and TGF-b1 and intracellular expression of FoxP3 on CD4+ cells by FACS. Live CD4+ cells were gated (using anti-CD4 antibody -PE-TR and 7-AAD) for the graphs shown in FIG. 10. See FIG. 10. These results show that selective engagement of CTLA-4 by its preferential ligand B7.1 can induce antigen specific TGF-beta 1 producing suppressor/regulatory T cells. Therefore, ligands for CTLA-4 used in the artificial antigen presenting system along with MHC-peptide can produce antigen specific regulatory T cells both in vitro or in vivo.

### Example 15

### Regulation of T Cell Activation by Microsphere Bound Negative Regulators

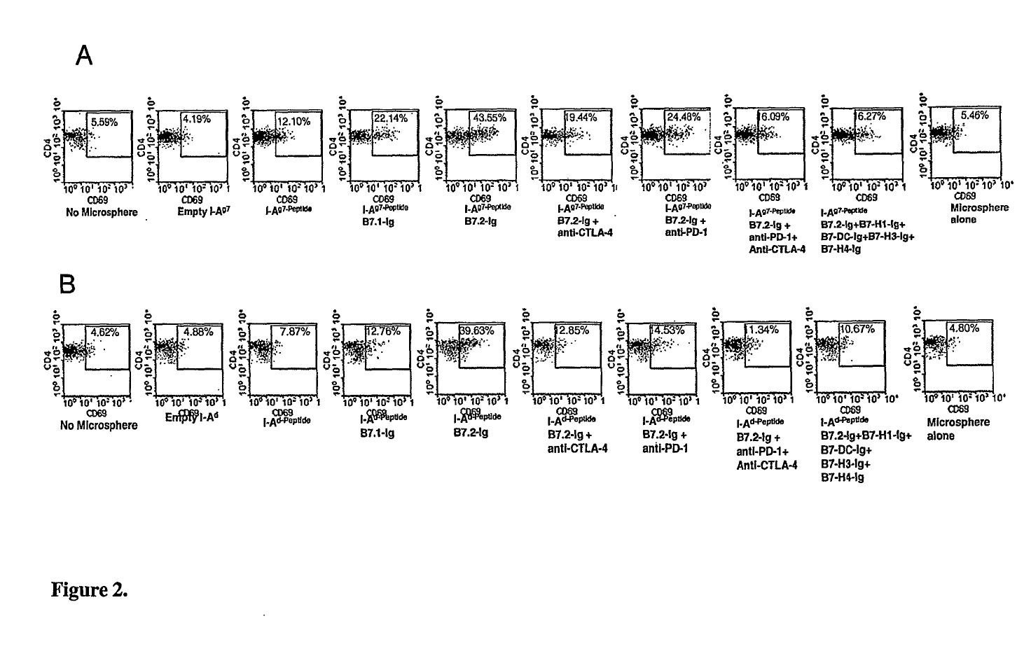

NOD.BDC2.5 TCR transgenic (A) and D011.10 TCR transgenic (B) mice (Balb/c mice) were primed with specific peptides ISQAVHAAHAEINEAGR (SEQ ID NO:1) and YVRPLWVRME (SEQ ID NO:2) peptide respectively intravenously along with LPS. Mice were sacrificed 3 days after injection and spleen cells were collected, CD4+ T cells were isolated by negative selection and cultured along with microspheres carrying different MHCs and ligands as mentioned below each panel. Cells that were cultured without microspheres and with blank (non-coated) microspheres are the extreme end panels. Cells were collected at 24 h culture and stained with FITC labeled CD69 and PE-labeled CD4 antibodies and tested by FACS for activation marker. I-Ag7 and I-Ad are MHC class molecules of NOD and Balb/c mice. Recombinant I-Ad-peptide contains the peptide sequence ISQAVHAAHAEINEAGR (SEQ ID NO: 1), and I-Ag7-peptide contains the peptide sequence YVRPLWVRME (SEQ ID NO:2) as antigens. See FIG. 2. These results indicate that artificial antigen presenting system can suppress antigen specific T cell activation when negative regulatory ligands are used in the presence or absence of costimulatory molecules. Further, MHC molecules with peptide sequence linked at cDNA level or empty MHC molecules passively loaded or covalently linked with antigenic peptides can be used in the biodegradable artificial antigen presenting system.

While the principals of this invention have been described in connection with specific embodiments, it should be understood clearly that these descriptions are added only by way of example and are not intended to limit, in any way, the scope of this invention. Other advantages and features of this invention will become apparent from the claims hereinafter, with the scope of those claims determined by their reasonable equivalents, as would be understood by those skilled in the art.

I claim:

1. An artificial antigen presenting microsphere, comprising (a) a biodegradable microsphere, (b) at least one MHC molecule coated onto the microsphere; and (c) at least one T-cell receptor ligand coated onto the microsphere.

2. The artificial antigen presenting microsphere of claim 1, wherein the at least one T-cell receptor ligand is an inhibitory ligand.

3. The artificial antigen presenting microsphere of claim 1, wherein the T-cell receptor ligand is a stimulatory ligand.

4. The artificial antigen presenting microsphere of claim 1, wherein the microsphere comprises an inhibitory ligand and a stimulatory ligand coated onto the microsphere.

5. The artificial antigen presenting microsphere of claim 1, wherein the microsphere is between about 0.2 μm and 500 μm in diameter.

6. The artificial antigen presenting microsphere of claim 4, wherein the microsphere is about 20 μm in diameter.

7. The artificial antigen presenting microsphere of claim 1, wherein the biodegradable microsphere is a biodegradable polymer.

8. The biodegradable microsphere of claim 7, wherein the polymer is selected from the group consisting of polylactide, poly(lactic acid-co-glycolic acid), poly(dioxanone), poly(trimethylene carbonate) copolymer, poly(caprolactone) homopolymer, polyanhydride, polyorthoester, polyphosphazene, poly(caprolactone) copolymer, alginate, PEO, Poly((lactide-co-ethyleneglycol)-co-ethyloxyphosphate) Poly(LAEG-EOP), Poly(1,4-bis(hydroxyethyl)terephthalate-co-ethyloxyphosphate) Poly(BHET-EOP), Poly(1,4-bis(hydroxyethyl)terephthalate-alt-ethyloxyphosphate)-co-1,4-bis(hydroxyethyl)terephthalate-co-terephthalate), Poly(BHET-EOP/TC, 80/20), and PMMA (Polymethylmethacrylate).

9. The artificial antigen presenting microsphere of claim 8, wherein the biodegradable polymer is a polylactide.

10. The artificial antigen presenting microsphere of claim 1, wherein the biodegradable microsphere is a polysaccharide.

11. The artificial antigen presenting microsphere of claim 1, wherein the biodegradable microsphere is a protein.

12. The artificial antigen presenting microsphere of claim 11, wherein the protein is selected from the group consisting of collagen, albumin, gelatin, elastin, and silk fibroin.

13. The artificial antigen presenting microsphere of claim 1, wherein the biodegradable microsphere is a lipid microsphere.

14. The artificial antigen presenting microsphere of claim 13, wherein the lipid microsphere is modified with one or more binding proteins on its surface.

15. The artificial antigen presenting microsphere of claim 14, wherein the one or more binding molecules is selected from the group consisting of protein A, protein L, protein G, protein A/G, streptavidin, avidin, extravidin, biotin, and antibodies.

16. The artificial antigen presenting microsphere of claim 1, wherein the microsphere further comprises (d) one or more molecules that can stabilize an interaction between a T cell receptor and the one or more MHC:antigens coated on the microsphere.

17. The artificial antigen presenting microsphere of claim 1, wherein the antigen is a self antigen.

18. The artificial antigen presenting microsphere of claim 17, wherein the self antigen is selected from the group consisting of antigenic peptides of insulin, insulin beta, GAD, GAD65, HSP, thyroglobulin, nuclear proteins, acetylcholine receptor, collagen, TSHR, ICA512(IA-2) and IA-2β (phogrin), carboxypeptidase H, ICA69, ICA12, and thyroid peroxidase.

19. The artificial antigen presenting microsphere of claim 1, wherein the antigen is an allergenic protein peptide.

20. The artificial antigen presenting microsphere of claim 1, wherein the MHC is selected from the group consisting of MHC I and MHC II.

21. The artificial antigen presenting microsphere of claim 1, wherein the MHC carries an antigen (MHC:antigen).

22. The artificial antigen presenting microsphere of claim 1, wherein the MHC in the MHC:antigen complex is selected from the group consisting of MHC I and MHC II.

23. The artificial antigen presenting microsphere of claim 2, wherein the at least one inhibitory ligand is selected from the group consisting of B7.1wa, CTLA-4 binding proteins, PD-L1, HVEM, PDL-2, B7-H3, B7-H4, OX-2, TGF-beta1, IL-10, IL-4, natural, recombinant and artificial ligands for CTLA-4, PD-1, OX-2 receptor, B7-H3 receptor, B7-H4 receptor and BTLA, natural and recombinant anti-CTLA-4 agonistic antibody, anti-PD-1 agonistic antibody, anti BTLA agonistic Antibody Anti-B7-H3 receptor agonistic antibody, Anti-B7-H4 receptor antibody, anti-CD28 antagonistic antibody, and anti-ICOS antagonistic antibody.

24. The artificial antigen presenting microsphere of claim 4, wherein the stimulatory ligand is selected from the group consisting of B7.1, B7.2, ICOS-L, Ox40-L, IL-2, IL-7, IFN-gamma, IL-12, IL-15, IL-17, IL-18, IL-22, TNF-alpha, LFA-3, ICAM-1, anti-CD28 agonistic antibody, anti-CTLA-4 antagonistic antibody, anti-ICOS agonistic antibody, anti-PD-1-antagonistic antibody, anti-B7-H3 receptor antagonistic antibody, anti-B7-H4 receptor antagonistic antibody, anti-BTLA-antagonistic antibody, natural, recombinant and artificial ligands for CD28, ICOS, OX-40, IL-2R, IL-7R, IFN-gamma R.

25. The artificial antigen presenting microsphere of claim 1, wherein the microsphere is modified to carry one or more active chemical groups, wherein the one or more chemical groups are selected from the group consisting of a sulf-hydryl, a carbo-hydryl, an amine, a carboxyl, and a hydroxyl.

26. The artificial antigen presenting microsphere of claim 2, wherein the inhibitory ligand is a death inducing ligand.

27. The artificial antigen presenting microsphere of claim 2, wherein the death inducing ligand is selected from the group consisting of FAS-L, TRAIL, anti-FAS agonistic antibody, and anti-TRAIL-agonistic antibody.

28. The artificial antigen presenting microsphere of claim 4, wherein the inhibitory ligand is a death inducing ligand.

29. The artificial antigen presenting microsphere of claim 28, wherein the death inducing ligand is selected from the group consisting of FAS-L, TRAIL, anti-FAS agonistic antibody, and anti-TRAIL-agonistic antibody.

30. The artificial antigen presenting microsphere of claim 1, wherein the at least one T-cell receptor ligand adheres to the microsphere via a binding molecule attached to the surface of the microsphere.

31. The artificial antigen presenting microsphere of claim 30, wherein the binding molecule is selected from the group consisting of protein A, biotin, avidin, extravidin, streptavidin, Protein G, protein L, protein A/G, antibody, anti-antibody.

32. A method for inducing antigen specific tolerance, wherein the method comprises contacting a T-cell with the microsphere of claim 1, wherein at least one MHC molecule and at least one T-cell receptor ligand are coated onto the microsphere.

33. A method for treating a patient suffering from an autoimmune disease comprising: obtaining T cells from a patient, culturing or incubating the T cells with a composition comprising an artificial antigen presenting microsphere of claim 1, expanding the T cells, and administering a pharmaceutical composition comprising the expanded T-cell population to the patient.

34. A method of treating a patient suffering from an autoimmune disease, comprising: administering a pharmaceutical composition comprising the artificial antigen presenting microsphere of claim 1.

35. The method of claim 34, wherein the autoimmune disease is selected from the group consisting of autoimmune gastritis, diabetes, autoimmune hemolytic anemia, autoimmune neutropenia, pernicious anemia, polyarteritis nodosa systemic lupus erythematosus, Wegener's granulomatosis, Autoimmune hepatitis, Behçet's disease, Crohn's disease, Primary bilary cirrhosis, Scleroderma, Ulcerative colitis, Sjögren's syndrome, Type 1 diabetes mellitus, Uveitis, Graves' disease, Thyroiditis, Type 1 diabetes mellitus, Myocarditis, Rheumatic fever, Scleroderma, Ankylosing spondylitis, Rheumatoid arthritis, Glomerulonephritis, Systemic lupus erythematosus, Type 1 diabetes mellitus, Rheumatoid arthritis, Sarcoidosis, Scleroderma, Systemic lupus erythematosus, Dermatomyositis, Myasthenia gravis, Polymyositis, Guillain-Barré syndrome, Multiple sclerosis, Systemic lupus erythematosus, Alopecia greata, Pemphigus/pemphigoid, Psoriasis, Scleroderma, Systemic lupus erythematosus, and Vitiligo.

36. A method for expanding protective antigen specific T cells, wherein the method comprises obtaining T cells from a patient, culturing or incubating the T cells with a composition comprising an artificial antigen presenting microsphere and expanding the T cells.

37. The method of claim 36, wherein the culturing or incubating of the T cells with a composition comprising an artificial antigen presenting microsphere is in the presence of one or more soluble factors.

38. A method for inducing differentiation of T cells, wherein the method comprises obtaining T cells from a patient, culturing or incubating the T cells with a composition comprising the artificial antigen presenting microsphere of claim 1, and converting to a differentiated T cell.

39. The method of claim 38, wherein the differentiated T cell is selected from the group consisting of suppressor T cells, regulatory T cells, effector T cells, specific cytokine producing T cells, and cytotoxic T cells.

40. The method of claim 36, wherein the microsphere is biodegradable.

41. The method of claim 37, wherein the one or more soluble factors is selected from the group consisting of cytokine(s), chemokine(s), and antibodies.


SEQUENCE LISTING (2 sequences)

SEQ ID NO: 1 - protein, 17 aa, Artificial peptide
  Synthetic
ISQAVHAAHAEINEAGR

SEQ ID NO: 2 - protein, 10 aa, Artificial peptide
  Synthetic
YVRPLWVRME